(19) United States
(12) Patent Application Publication
(10) Pub. No.: US 2015/0191798 A1
(43) Pub. Date: Jul. 9, 2015

(54) HOXC6 AND OVARIAN CANCER METHODS AND USES THEREOF

(71) Applicants: The University of North Carolina at Charlotte, Charlotte, NC (US); The Charlotte-Mecklenburg Hospital Authority d/b/a Carolinas Healthcare System, Charlotte, NC (US)

(72) Inventors: M. Taghi Mostafavi, Charlotte, NC (US); Christine Richardson, Charlotte, NC (US); Zahra Bahrani-Mostafavi, Charlotte, NC (US); David L. Tait, Charlotte, NC (US)

(21) Appl. No.: 14/662,649

(22) Filed: Mar. 19, 2015

Related U.S. Application Data
(63) Continuation-in-part of application No. 14/168,271, filed on Jan. 30, 2014.
(60) Provisional application No. 61/760,806, filed on Feb. 5, 2013.

(51) Int. Cl. C12Q 1/68; G01N 33/574

(52) U.S. Cl. CPC C12Q 1/6886; G01N 33/57449; C12Q 2600/16; C12Q 2600/118; G01N 2333/4703

(57) ABSTRACT

The present invention comprises novel methods, systems, devices, and kits to detect cancer in a patient using HOXC6. Methods and systems are presented herein to (1) determine differential gene expression in cancer tissue (e.g. human epithelial ovarian cancer) using Exon microarray analysis and confirm select gens using qPCR; (2) to correlate transcriptional expression from part 1 with potential protein using IHC; and (3) to confirm specific proteins in sera by ELISA process. In some embodiments, a ELISA kit is provided. More specifically, the inventors developed a cancer screen test based on significant changes in HOXC6 protein in blood serum of ovarian cancer human subjects. Industry available standard protocols and reagents for the detection of HOXC6 in patient blood serum provided highly variable results that would be unsatisfactory for clinical diagnostic testing and screening. Thus the inventors developed and optimized a protocol for indirect sandwich ELISA to detect the HOXC6 protein in blood serum suitable for clinical use.

### CROSS REFERENCE TO RELATED APPLICATIONS

This application claims the benefit of U.S. application Ser. No. 14/168,271 filed Jan. 30, 2014, Mostafavi et al., Atty. Docket No. 2013-019UTL1 and U.S. Provisional Application No. 61/760,806 filed Feb. 5, 2013, Mostafavi et al., Atty. Docket No. 2013-019PRO1 which are hereby incorporated by reference in their entirety.

### 1. FIELD OF THE INVENTION

The present invention relates to the field cancer detection. More specifically, the present invention relates to novel methods, systems, devices, and kits to detect ovarian cancer in a patient using HOXC6 expression levels.

### 2. BACKGROUND OF THE INVENTION

2.1. Introduction

Ovarian cancer is the fifth leading cause of cancer death among women in the United States. More than 21,000 new cases are diagnosed in the United States each year. Five-year survival of women diagnosed with stage I-IV disease is 88% (as high as 94% for women diagnosed in the earliest stage I), 66%, 34%, and 18%, respectively. Overall, ovarian cancer is so deadly because early ovarian cancer, e.g., stage I, is typically asymptomatic, and 75% of new cases are only diagnosed when the cancer has reached the advanced stages. Survival for these advance stage patients is only 30-40%. Heintz et al., Carcinoma of the ovary. Int J Gynaecol Obstet 2003, 83 Suppl 1:135-166. Research continues to focus on determining the gene expression pathways associated with ovarian cancer.

Pathways of tumor development have been extensively researched leading to the elucidation of some major paradigms in cancer biology. Transcription factors, an important focus of cancer research, function in malignant transformation, as therapeutic targets, or as biomarkers. Transcription factors are DNA binding proteins that bind to regulatory regions of specific genes, thereby activating transcription of the target genes. These proteins are organized into many classes characterized by homologous regions within their DNA binding domains. Typically localized to the nucleus, their function is activated through different cell signaling cascades. Fekete et al., Meta-analysis of gene expression profiles associated with histological classification and survival in 829 ovarian cancer samples. Int J Cancer 2012, 131(1):95-105.

One group of transcription factors that has become a subject of interest in cancer biology is the human homeobox class I (HOX) group. There are 39 HOX genes clustered in four chromosomal loci in humans and expression of each HOX gene is tightly regulated. Alexander et al., Hox genes and segmentation of the hindbrain and axial skeleton. Annu Rev Cell Dev Biol 2009, 25:431-456. These genes direct cell differentiation and maintenance of cell identity and morphology from early embryonic development throughout adulthood. Research has shown altered expression levels of many HOX genes in several cancers, including endometrial, cervical, pancreatic, thyroid, and lung. Calvo et al., Altered HOX and WNT7A expression in human lung cancer. Proceedings of the National Academy of Sciences of the United States of America 2000, 97(23):12776-12781; Flagiello et al., Relationship between DNA methylation and gene expression of the HOXB gene cluster in small cell lung cancers. FEBS Lett 1996, 380(1-2):103-107; Hung et al., Homeobox gene expression and mutation in cervical carcinoma cells. Cancer Sci 2003, 94(5):437-441; Lane et al., HOXA10 expression in endometrial adenocarcinoma. Tumour Biol 2004, 25(5-6):264-269; Segara et al., Expression of HOXB2, a retinoic acid signaling target in pancreatic cancer and pancreatic intraepithelial neoplasia. Clin Cancer Res 2005, 11(9):3587-3596; Takahashi et al., Expression profiles of 39 HOX genes in normal human adult organs and anaplastic thyroid cancer cell lines by quantitative real-time RT-PCR system. Exp Cell Res 2004, 293(1):144-153.

HOXC6 expression has been reported in meduloblastomas, osteosarcomas, breast, lung, gastrointestinal, head and neck squamous, and prostate carcinomas. Alexander et al. 2009; Bodey et al., Homeobox B3, B4, and C6 gene product expression in osteosarcomas as detected by immunocytochemistry. Anticancer Res 2000, 20(4):2717-2721; Castronovo et al., Homeobox genes: potential candidates for the transcriptional control of the transformed and invasive phenotype. Biochem Pharmacol 1994, 47(1):137-143; Fujiki et al., Hoxc6 is overexpressed in gastrointestinal carcinoids and interacts with JunD to regulate tumor growth. Gastroenterology 2008, 135(3):907-916, 916 e901-902; Miller et al., Aberrant HOXC expression accompanies the malignant phenotype in human prostate. Cancer Res 2003, 63(18):5879-5888; Moon et al., HOXC6 is deregulated in human head and neck squamous cell carcinoma and modulates Bcl-2 expression. The Journal of biological chemistry 2012, 287(42):35678-35688. Although altered expression of several HOX genes has also been demonstrated in ovarian cancer, little is known about HOXC6 expression in ovarian cancer. Bahrani-Mostafavi et al., Correlation analysis of HOX, ErbB and IGFBP family gene expression in ovarian cancer. Cancer Invest 2008, 26(10):990-998; Naora et al., Aberrant expression of homeobox gene HOXA7 is associated with mullerian-like differentiation of epithelial ovarian tumors and the generation of a specific autologous antibody response. Proceedings of the National Academy of Sciences of the United States of America 2001, 98(26):15209-15214; Naora et al., serologically identified tumor antigen encoded by a homeobox gene promotes growth of ovarian epithelial cells. Proceedings of the National Academy of Sciences of the United States of America 2001, 98(7):4060-4065.

Currently, there are no reliable screening tests for early detection despite intense interest in their development. Evaluation of CA125 along with ultrasound has been used clinically in high-risk populations; however, several trials evaluating CA125, ultrasound, and serum marker panels failed meet statistical criteria as effective early detection tests. Studies of multiple biomarker panels will improve screening sensitivity and specificity for better and acceptable test.

### 3. SUMMARY OF THE INVENTION

In particular non-limiting embodiments, the present invention provides a method for detecting the likelihood of ovarian cancer which comprises: measuring a level of HOXC6 in a blood or tissue sample; and if the level of HOXC6 is 50% or less than a normal level of HOXC6, determining that the blood or tissue sample indicates an increased likelihood of having ovarian cancer.

The level of HOXC6 may be measured using an antibody-based assay such as an indirect enzyme-linked immunesorbant assay (ELISA). The blood sample may be a blood serum sample.

The invention also provides a kit comprising: at least one reagent selected from the group consisting of: a primary antibody capable of specifically binding a HOXC6 protein; a secondary antibody capable of detecting the primary antibody bound to the HOXC6 protein; and instructions for use in measuring a level of HOXC6 from a subject suspected of having ovarian cancer wherein levels of 50% or less than a normal level, determining that the subject has increased likelihood of having ovarian cancer. In the methods and kits above, increased likelihood of ovarian cancer may be associated with levels of 60% or less than normal, 65% or less than normal, 70% or less than normal, 75% or less than normal, 80% or less than normal, 85% or less than normal, 90% or less than normal, or 95% or less than normal.

The invention also provides a method of identifying a compound that may become a therapeutic target for the treatment of ovarian cancer, the method comprising the steps of: contacting a compound with a sample comprising a cell or a tissue; measuring a level of HOXC6 in the cell or tissue; and determining a functional effect of the compound on the level of HOXC6; thereby identifying a compound that prevents or treats ovarian cancer.

The present invention comprises novel methods, systems, devices, and kits to detect cancer in a patient using HOXC6. Methods and systems are presented herein to (1) determine differential gene expression in cancer tissue (e.g. human epithelial ovarian cancer) using Exon microarray analysis and confirm select gens using qPCR; (2) to correlate transcriptional expression from part 1 with potential protein using IHC; and (3) to confirm specific proteins in sera by ELISA process. In some embodiments, an ELISA kit is provided. More specifically, the inventors developed a cancer screen test based on significant changes in HOXC6 protein in blood serum of ovarian cancer human subjects. Industry available standard protocols and reagents for the detection of HOXC6 in patient blood serum provided highly variable results that would be unsatisfactory for clinical diagnostic testing and screening. Thus the inventors developed and optimized a protocol for indirect sandwich ELISA to detect the HOXC6 protein in blood serum suitable for clinical use.

The invention also includes the use of down-regulation of HOXC6 in combination with established markers such as B7-H4 (Simon I, et al. B7-H4 is over-expressed in early-stage ovarian cancer and is independent of CA125 expression. Gynecologic Oncology. 2007; 106(2):334-341, Shah C A, et al. Influence of ovarian cancer risk status on the diagnostic performance of the serum biomarkers mesothelin, HE4, and CA125. Cancer Epidemiology Biomarkers and Prevention. 2009; 18(5):1365-1372); BRCA1/BRCA2 (Chen S, et al. Characterization of BRCA1 and BRCA2 mutations in a large United States sample. Journal of Clinical Oncology. 2006; 24(6):863-871), CA-125, HE4 alone or in combination with CA125 (Molina R, et al. HE4 a novel tumour marker for ovarian cancer: comparison with CA 125 and ROMA algorithm in patients with gynaecological diseases. Tumour Biology. 2011; 32(6):1087-1095, Montagnana, et al. HE4 in ovarian cancer: from discovery to clinical application. Advances in Clinical Chemistry. 2011; 55:1-20, Moore R G, et al. A novel multiple marker bioassay utilizing HE4 and CA125 for the prediction of ovarian cancer in patients with a pelvic mass. Gynecologic Oncology. 2009; 112(1):40-46); KLK6 (Diamandis E P, et al. Human kallikrein 6 (hK6): a new potential serum biomarker for diagnosis and prognosis of ovarian carcinoma. Journal of Clinical Oncology. 2003; 21(6):1035-1043); osteopontin (Kim J H, et al. Osteopontin as a potential diagnostic biomarker for ovarian cancer. The Journal of the American Medical Association. 2002; 287(13):1671-1679), and/or prostasin (Mok S C, et al. Prostasin, a potential serum marker for ovarian cancer: identification through microarray technology. Journal of the National Cancer Institute. 2001; 93(19): 1458-1464).

The invention also includes the use two, three or more of the markers in Table 3 of the specification.

### 4. BRIEF DESCRIPTION OF THE FIGURES

FIG. 1 shows transcriptional microarray analysis of 37 HOX genes in serous ovarian carcinomas. Shown are relative expression levels of 37 HOX genes from two normal and five malignant samples. Unsupervised clustering analysis (hierarchical tree shown on left side) separated normal samples (NNE, grey bar) from malignant tumor samples (TM, white bar).

FIG. 2 illustrates an example of quantitative RT-PCR analysis of HOXC6 expression in serous ovarian carcinomas. Increased amounts of HOXC6 mRNA was detected in normal epithelium and decreased amounts of HOXC6 mRNA was detected in malignant tumor cells as normalized to normal human ovarian surface epithelial cell line (HOSE) reference sample [32]. Data are presented as means and standard deviation of three replicates for each. ** indicates statistically significant difference calculated between sample and HOSE standard (M1 p-value=0.0002, M2 p-value=0.004, M3 p-value=0.0030).

FIG. 3 shows the presence and localization of HOXC6 protein in normal ovarian surface epithelium but not in malignant tumor. (Panel A) Hematoxylin & eosin-Normal ovary. (Panel B) HoxC6-Normal ovary. (Panel C) Hematoxylin and eosin-Serous ovarian carcinoma. (Panel D) HoxC6-Serous ovarian carcinoma. All sections shown at 200×.

FIG. 4 shows quantitative RT-PCR analysis of HOXC6 expression in serous ovarian carcinoma. Data are presented as means and standard deviation of three replicates for each. ** indicates statistically significant difference calculated between sample and HOSE 6-3 standard (M1 p-value=0.0002, M2 p-value=0.004, M3 p-value=0.0030), using the comparative Ct method for quantification of all mRNA transcripts [33].

FIG. 5 shows ELISA analysis of HOXC6 protein in serum. Sera were assayed in triplicates and OD values and the standard curve used to calculate pg/mL concentrations of each. Non-malignant normal samples (normal) shown on left. Malignant samples (malignant) shown on right. The difference between groups was statistically significant (p-value=1.18×10⁻¹³).

### 5. DETAILED DESCRIPTION OF THE INVENTION

This invention is also directed to a method of diagnosing ovarian cancer in a sample from a subject comprising: (a) detecting HOXC6 in a sample obtained from the subject, by an antibody assay with antibodies specific for HOXC6; (b) comparing the detected levels to at least one sample from a training set(s), wherein a sample training set(s) comprises data from the levels from a reference sample, and the comparing step comprises applying a statistical algorithm which comprises determining a correlation between the detected levels from the subject and the detected levels from at least one training set(s); and (c) diagnosing ovarian cancer of the subject based on the detected levels from the subject and the results of the statistical algorithm.

In one embodiment, the ovarian cancer is serous ovarian carcinoma.

### 5.1. Definitions

“Labeled,” “labeled with a detectable label,” and “detectably labeled” are used interchangeably herein to indicate that an entity (e.g., a probe) can be detected. “Label” and “detectable label” mean a moiety attached to an entity to render the entity detectable, such as a moiety attached to a probe to render the probe detectable upon binding to a target sequence. The moiety, itself, may not be detectable but may become detectable upon reaction with yet another moiety. Use of the term “detectably labeled” is intended to encompass such labeling. The detectable label can be selected such that the label generates a signal, which can be measured and the intensity of which is proportional to the amount of bound entity. A wide variety of systems for labeling and/or detecting molecules, such as nucleic acids, e.g., probes, are well-known. Labeled nucleic acids can be prepared by incorporating or conjugating a label that is directly or indirectly detectable by spectroscopic, photochemical, biochemical, immunochemical, electrical, optical, chemical or other means. Suitable detectable labels include radioisotopes, fluorophores, chromophores, chemiluminescent agents, microparticles, enzymes, magnetic particles, electron dense particles, mass labels, spin labels, haptens, and the like. Fluorophores and chemiluminescent agents are preferred herein.

“Predetermined cutoff” and “predetermined level” refer generally to a cutoff value that is used to assess diagnostic/prognostic/therapeutic efficacy results by comparing the assay results against the predetermined cutoff/level, where the predetermined cutoff/level already has been linked or associated with various clinical parameters (e.g., severity of disease, progression/nonprogression/improvement, etc.).

The term “sensitivity” as used herein refers to the number of true positives divided by the number of true positives plus the number of false negatives, where sensitivity (“sens”) may be within the range of 0<sens<1. Ideally, method embodiments herein have the number of false negatives equaling zero or close to equaling zero, so that no subject is wrongly identified as not having ovarian cancer when they indeed have ovarian cancer. Conversely, an assessment often is made of the ability of a prediction algorithm to classify negatives correctly, a complementary measurement to sensitivity. The term “specificity” as used herein refers to the number of true negatives divided by the number of true negatives plus the number of false positives, where specificity (“spec”) may be within the range of 0<spec<1. Ideally, the methods described herein have the number of false positives equaling zero or close to equaling zero, so that no subject is wrongly identified as having ovarian cancer when they do not in fact have ovarian cancer. Hence, a method that has both sensitivity and specificity equaling one, or 100%, is preferred.

The phrase “functional effects” in the context of assays for testing means compounds that modulate a phenotype or a gene associated with ovarian cancer either in vitro, in cell culture, in tissue samples, or in vivo. This may also be a chemical or phenotypic effect such as altered HOXC6 profiles in vivo, e.g., changing from a high risk of HOXC6 profile to a low risk profile; altered expression of genes associated with ovarian cancer; altered transcriptional activity of a gene hyper- or hypomethylated in ovarian cancer; or altered activities and the downstream effects of proteins encoded by these genes. A functional effect may include transcriptional activation or repression, the ability of cells to proliferate, expression in cells during ovarian cancer progression, and other cellular characteristics. “Functional effects” include in vitro, in vivo, and ex vivo activities. By “determining the functional effect” is meant assaying for a compound that increases or decreases the transcription of genes or the translation of proteins that are indirectly or directly associated with ovarian cancer. Such functional effects can be measured by any means known to those skilled in the art, e.g., changes in spectroscopic characteristics (e.g., fluorescence, absorbance, refractive index); hydrodynamic (e.g., shape), chromatographic; or solubility properties for the protein; ligand binding assays, e.g., binding to antibodies; measuring inducible markers or transcriptional activation of the marker; measuring changes in enzymatic activity; the ability to increase or decrease cellular proliferation, apoptosis, cell cycle arrest, measuring changes in cell surface markers. Validation of the functional effect of a compound on ovarian cancer occurrence or progression can also be performed using assays known to those of skill in the art such as studies using mouse models. The functional effects can be evaluated by many means known to those skilled in the art, e.g., microscopy for quantitative or qualitative measures of alterations in morphological features, measurement of changes in RNA or protein levels for other genes associated with ovarian cancer, measurement of RNA stability, identification of downstream or reporter gene expression (CAT, luciferase, β-gal, GFP, and the like), e.g., via chemiluminescence, fluorescence, colorimetric reactions, antibody binding, inducible markers, etc.

The term “HOXC6” as used herein refers to a human homeobox protein Hox-C6, size: 235 amino acids; 26915 Da; Gene ID 3223; UniProt P09630; REFSEQ proteins (2 alternative transcripts): NP_{—}004494.1, NP_{—}710160.1; ENSEMBL proteins: ENSP00000424124, ENSP00000423898, ENSP00000377864, ENSP00000243108. Mouse, goat, rabbit ponoclonal and polyclonal antibodies to HOXC6 are available from a variety of sources including antibodies-online Inc. (Atlanta, Ga.). Examples of include antibodies to the full length protein, the C-terminal, e.g, AA 208-238, AA 200-227; N-terminal AA 22-36. The antibodies may be conjugated with a dye, e.g., Alexa Fluor 350, 488, 555, 647; Cy 5, 5.5, 7; or FITC; an enzyme, e.g., alkaline phosphatase; or other marker, e.g., biotin.

“Inhibitors,” “activators,” and “modulators” of the markers are used to refer to activating, inhibitory, or modulating molecules identified using in vitro and in vivo assays of the expression of genes hyper- or hypomethylated in ovarian cancer, mutations associated with ovarian cancer, or the translation proteins encoded thereby Inhibitors, activators, or modulators also include naturally occurring and synthetic ligands, antagonists, agonists, antibodies, peptides, cyclic peptides, nucleic acids, antisense molecules, ribozymes, shRNAs, RNAi molecules, small organic molecules and the like. Such assays for inhibitors and activators include, e.g., (1)(a) the mRNA expression, or (b) proteins expressed by genes hyper- or hypomethylated in ovarian cancer in vitro, in cells, or cell extracts; (2) applying putative modulator compounds; and (3) determining the functional effects on activity, as described above.

Assays comprising in vivo measurement of ovarian cancer; or genes hyper- or hypomethylated in ovarian cancer are treated with a potential activator, inhibitor, or modulator are compared to control assays without the inhibitor, activator, or modulator to examine the extent of inhibition. Controls (untreated) are assigned a relative activity value of 100% Inhibition of gene expression, protein expression associated with ovarian cancer is achieved when the activity value relative to the control is about 80%, preferably 50%, more preferably 25-0%. Activation of gene expression, or proteins associated with ovarian cancer is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide, small organic molecule, polysaccharide, peptide, circular peptide, lipid, fatty acid, shRNA, siRNA, polynucleotide, oligonucleotide, etc., to be tested for the capacity to directly or indirectly modulate a genotype or phenotype associated with ovarian cancer. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (“HTS”) methods are employed for such an analysis. The compound may be a “small organic molecule” that is an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

Antibodies

Another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)2 fragments which can be generated by treating the antibody with an enzyme such as pepsin. Alternatively, monomeric binders such as scFv, diabodies, minibodies, small immunoproteins (SIPs) may be prepared. Olafsen et al. 2005 Cancer Res 65:5907-5916; Borsi et al. 2002 Int J Cancer 102:75-85; Berndorff et al. 2005 Clin Cancer Res 11:7053s-7063s; and Tijink et al. 2006 J Nucl Med 47:1127-1135. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Antibody-producing cells can be obtained from the subject and used to prepare monoclonal antibodies by standard techniques, such as the hybridoma technique originally described by Kohler and Milstein 1975 Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409 (Winter); PCT Publication Nos. WO 92/18619; WO 91/17271; WO 92/20791; WO 92/15679; WO 93/01288; WO 92/01047; WO 92/09690; WO 90/02809; Fuchs et al. 1991 Bio/Technology 9:1370-1372; Hay et al. 1992 Hum. Antibod. Hybridomas 3:81-85; Huse et al. 1989 Science 246:1215-1281; Griffiths et al. 1993 EMBO J. 12:725-734.

An antibody directed against a polypeptide corresponding to a marker of the invention (e.g., a monoclonal antibody) can be used to isolate the polypeptide by standard techniques, such as affinity chromatography or immunoprecipitation. Moreover, such an antibody can be used to detect the marker (e.g., in a cellular lysate or cell supernatant) in order to evaluate the level and pattern of expression of the marker. The antibodies can also be used diagnostically to monitor protein levels in tissues or body fluids (e.g., in a tumor cell-containing body fluid) as part of a clinical testing procedure, e.g., to for example, determine the efficacy of a given treatment regimen. Detection can be facilitated by coupling the antibody to a detectable substance. Examples of detectable substances include, but are not limited to, various enzymes, prosthetic groups, fluorescent materials, luminescent materials, bioluminescent materials, and radioactive materials. Examples of suitable enzymes include, but are not limited to, horseradish peroxidase, alkaline phosphatase, β-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to, luciferase, luciferin, and aequorin, and examples of suitable radioactive materials for diagnostics or therapeutics include, but are not limited to, ³H, ¹²⁵I, ¹³¹I, ¹¹¹In, ¹⁷⁷Lu, ⁹⁰Y, or ³⁵S.

Statistical Methods

The data may be ranked for its ability to distinguish biomarkers in both the 1 versus all (i.e., disease versus normal) and the all-pairwise (i.e., normal versus specific disease) cases. One statistic used for the ranking is the area under the receiver operator characteristic (ROC) curve (a plot of sensitivity versus (1-specificity)). Although biomarkers are evaluated for reliability across datasets, the independent sample sets are not combined for the purposes of the ROC ranking. As a result, multiple independent analyses are performed and multiple independent rankings are obtained for each biomarker's ability to distinguish groups of interest.

It is to be understood that other genes and/or diagnostic criteria may be used in this invention. For example, patient characteristics, standard blood workups, the results of imaging tests, and/or histological evaluation may optionally be combined with biomarkers disclosed herein.

Such analysis methods may be used to form a predictive model, and then use that model to classify test data. For example, one convenient and particularly effective method of classification employs multivariate statistical analysis modeling, first to form a model (a “predictive mathematical model”) using data (“modeling data”) from samples of known class (e.g., from subjects known to have, or not have, a particular class, subclass or grade of lung cancer), and second to classify an unknown sample (e.g., “test data”), according to lung cancer status.

Pattern recognition (PR) methods have been used widely to characterize many different types of problems ranging for example over linguistics, fingerprinting, chemistry and psychology. In the context of the methods described herein, pattern recognition is the use of multivariate statistics, both parametric and non-parametric, to analyze spectroscopic data, and hence to classify samples and to predict the value of some dependent variable based on a range of observed measurements. There are two main approaches. One set of methods is termed “unsupervised” and these simply reduce data complexity in a rational way and also produce display plots which can be interpreted by the human eye. The other approach is termed “supervised” whereby a training set of samples with known class or outcome is used to produce a mathematical model and is then evaluated with independent validation data sets.

Unsupervised PR methods are used to analyze data without reference to any other independent knowledge. Examples of unsupervised pattern recognition methods include principal component analysis (PCA), hierarchical cluster analysis (HCA), and non-linear mapping (NLM).

Alternatively, and in order to develop automatic classification methods, it has proved efficient to use a “supervised” approach to data analysis. Here, a “training set” of biomarker expression data is used to construct a statistical model that predicts correctly the “class” of each sample. This training set is then tested with independent data (referred to as a test or validation set) to determine the robustness of the computer-based model. These models are sometimes termed “expert systems,” but may be based on a range of different mathematical procedures. Supervised methods can use a data set with reduced dimensionality (for example, the first few principal components), but typically use unreduced data, with all dimensionality. In all cases the methods allow the quantitative description of the multivariate boundaries that characterize and separate each class, for example, each class of lung cancer in terms of its biomarker expression profile. It is also possible to obtain confidence limits on any predictions, for example, a level of probability to be placed on the goodness of fit (see, for example, Sharaf; Illman; Kowalski, eds. (1986). Chemometrics. New York: Wiley). The robustness of the predictive models can also be checked using cross-validation, by leaving out selected samples from the analysis.

Examples of supervised pattern recognition methods include the following nearest centroid methods (Dabney 2005 Bioinformatics 21(22):4148-4154 and Tibshirani et al. 2002 Proc. Natl. Acad. Sci. USA 99(10):6576-6572); soft independent modeling of class analysis (SIMCA) (see, for example, Wold, (1977) Chemometrics: theory and application 52: 243-282.); partial least squares analysis (PLS) (see, for example, Wold (1966) Multivariate analysis 1: 391-420; Joreskog (1982) Causality, structure, prediction 1: 263-270); linear discriminant analysis (LDA) (see, for example, Nillson (1965). Learning machines. New York.); K-nearest neighbor analysis (KNN) (see, for example, Brown and Martin 1996 J Chem Info Computer Sci 36(3):572-584); artificial neural networks (ANN) (see, for example, Wasserman (1993). Advanced methods in neural computing. John Wiley & Sons, Inc; O'Hare & Jennings (Eds.). (1996). Foundations of distributed artificial intelligence (Vol. 9). Wiley); probabilistic neural networks (PNNs) (see, for example, Bishop & Nasrabadi (2006). Pattern recognition and machine learning (Vol. 1, p. 740). New York: Springer; Specht, (1990). Probabilistic neural networks. Neural networks, 3(1), 109-118); rule induction (RI) (see, for example, Quinlan (1986) Machine learning, 1(1), 81-106); and, Bayesian methods (see, for example, Bretthorst (1990). An introduction to parameter estimation using Bayesian probability theory. In Maximum entropy and Bayesian methods (pp. 53-79). Springer Netherlands; Bretthorst, G. L. (1988). Bayesian spectrum analysis and parameter estimation (Vol. 48). New York: Springer-Verlag); unsupervised hierarchical clustering (see for example Herrero 2001 Bioinformatics 17(2) 126-136). In one embodiment, the classifier is the centroid based method described in Mullins et al. 2007 Clin Chem 53(7):1273-9, which is herein incorporated by reference in its entirety for its teachings regarding disease classification.

It is often useful to pre-process data, for example, by addressing missing data, translation, scaling, weighting, etc. Multivariate projection methods, such as principal component analysis (PCA) and partial least squares analysis (PLS), are so-called scaling sensitive methods. By using prior knowledge and experience about the type of data studied, the quality of the data prior to multivariate modeling can be enhanced by scaling and/or weighting. Adequate scaling and/or weighting can reveal important and interesting variation hidden within the data, and therefore make subsequent multivariate modeling more efficient. Scaling and weighting may be used to place the data in the correct metric, based on knowledge and experience of the studied system, and therefore reveal patterns already inherently present in the data.

If possible, missing data, for example gaps in column values, should be avoided. However, if necessary, such missing data may replaced or “filled” with, for example, the mean value of a column (“mean fill”); a random value (“random fill”); or a value based on a principal component analysis (“principal component fill”). Each of these different approaches will have a different effect on subsequent PR analysis.

“Translation” of the descriptor coordinate axes can be useful. Examples of such translation include normalization and mean centering. “Normalization” may be used to remove sample-to-sample variation. Many normalization approaches are possible, and they can often be applied at any of several points in the analysis. “Mean centering” may be used to simplify interpretation. Usually, for each descriptor, the average value of that descriptor for all samples is subtracted. In this way, the mean of a descriptor coincides with the origin, and all descriptors are “centered” at zero. In “unit variance scaling,” data can be scaled to equal variance. Usually, the value of each descriptor is scaled by 1/StDev, where StDev is the standard deviation for that descriptor for all samples. “Pareto scaling” is, in some sense, intermediate between mean centering and unit variance scaling. In pareto scaling, the value of each descriptor is scaled by 1/sqrt(StDev), where StDev is the standard deviation for that descriptor for all samples. In this way, each descriptor has a variance numerically equal to its initial standard deviation. The pareto scaling may be performed, for example, on raw data or mean centered data.

“Logarithmic scaling” may be used to assist interpretation when data have a positive skew and/or when data spans a large range, e.g., several orders of magnitude. Usually, for each descriptor, the value is replaced by the logarithm of that value. In “equal range scaling,” each descriptor is divided by the range of that descriptor for all samples. In this way, all descriptors have the same range, that is, 1. However, this method is sensitive to presence of outlier points. In “autoscaling,” each data vector is mean centred and unit variance scaled. This technique is a very useful because each descriptor is then weighted equally and large and small values are treated with equal emphasis. This can be important for analytes present at very low, but still detectable, levels.

Several supervised methods of scaling data are also known. Some of these can provide a measure of the ability of a parameter (e.g., a descriptor) to discriminate between classes, and can be used to improve classification by stretching a separation. For example, in “variance weighting,” the variance weight of a single parameter (e.g., a descriptor) is calculated as the ratio of the inter-class variances to the sum of the intra-class variances. A large value means that this variable is discriminating between the classes. For example, if the samples are known to fall into two classes (e.g., a training set), it is possible to examine the mean and variance of each descriptor. If a descriptor has very different mean values and a small variance, then it will be good at separating the classes. “Feature weighting” is a more general description of variance weighting, where not only the mean and standard deviation of each descriptor is calculated, but other well-known weighting factors, such as the Fisher weight, are used.

The methods described herein may be implemented and/or the results recorded using any device capable of implementing the methods and/or recording the results. Examples of devices that may be used include but are not limited to electronic computational devices, including computers of all types. When the methods described herein are implemented and/or recorded in a computer, the computer program that may be used to configure the computer to carry out the steps of the methods may be contained in any computer readable medium capable of containing the computer program. Examples of computer readable medium that may be used include but are not limited to diskettes, CD-ROMs, DVDs, ROM, RAM, and other memory and computer storage devices. The computer program that may be used to configure the computer to carry out the steps of the methods and/or record the results may also be provided over an electronic network, for example, over the internet, an intranet, or other network.

The process of comparing a measured value and a reference value can be carried out in any convenient manner appropriate to the type of measured value and reference value for the discriminative gene at issue. “Measuring” can be performed using quantitative or qualitative measurement techniques, and the mode of comparing a measured value and a reference value can vary depending on the measurement technology employed. For example, when a qualitative colorimetric assay is used to measure expression levels, the levels may be compared by visually comparing the intensity of the colored reaction product, or by comparing data from densitometric or spectrometric measurements of the colored reaction product (e.g., comparing numerical data or graphical data, such as bar charts, derived from the measuring device). However, it is expected that the measured values used in the methods of the invention will most commonly be quantitative values. In other examples, measured values are qualitative. As with qualitative measurements, the comparison can be made by inspecting the numerical data, or by inspecting representations of the data (e.g., inspecting graphical representations such as bar or line graphs).

Protein biomarkers may be analyzed using standard high throughput clinical chemistry methods such as immunoturbimetric or immunonephric assays available from a variety of vendors. Non-limiting examples include Electrochemiluminescence immunoassay “ECLIA”, using the MODULAR ANALYTICS E 170 analyzer (Roche); immunoturbidimetric assay, using the COBAS INTEGRA INTEGRA 400 analyzer (Roche); immunoturbimentric assays on the Abbott c8000 analyzer; LX20 (Beckman-Coulter); rx Daytona Randox Laboratories. See Maki et al. 2009 Comparison of immunoturbidimetric and immunonephelometric assays for specific proteins Clin Biochem 42 1568-1571.

The process of comparing may be manual (such as visual inspection by the practitioner of the method) or it may be automated. For example, an assay device (such as a luminometer for measuring chemiluminescent signals) may include circuitry and software enabling it to compare a measured value with a reference value for a biomarker protein. Alternately, a separate device (e.g., a digital computer) may be used to compare the measured value(s) and the reference value(s). Automated devices for comparison may include stored reference values for the biomarker protein(s) being measured, or they may compare the measured value(s) with reference values that are derived from contemporaneously measured reference samples (e.g., samples from control subjects).

As will be apparent to those of skill in the art, when replicate measurements are taken, the measured value that is compared with the reference value is a value that takes into account the replicate measurements. The replicate measurements may be taken into account by using either the mean or median of the measured values as the “measured value.”

The invention also includes methods of identifying patients for particular treatments or selecting patients for which a particular treatment would be desirable or contraindicated.

The methods above may be performed by a reference laboratory, a hospital pathology laboratory or a doctor. The methods may be performed as a Laboratory Developed Test (LDT) in a Clinical Laboratory Improvement Amendments (CLIA) approved lab, or an FDA-cleared test such as a 510(K). The methods may be performed in a centralized testing labororatory or on a point-of-care (POC) device. The methods above may further comprise an algorithm and/or statistical analysis.

### 5.2. Samples

The invention provides compositions and kits for detecting and/or measuring types and levels of HOXC6 using DNA assays, antibodies specific for the polypeptides or nucleic acids specific for the polynucleotides. Kits for carrying out the diagnostic assays of the invention typically include, a suitable container means, (i) a probe that comprises an antibody or nucleic acid sequence that specifically binds to the marker polypeptides or polynucleotides of the invention; (ii) a label for detecting the presence of the probe; and (iii) instructions for how to measure the HOXC6. The kits may include several antibodies or polynucleotide sequences encoding polypeptides of the invention, e.g., a first antibody and/or second and/or third and/or additional antibodies that recognize a protein or peptide associated with ovarian cancer. The container means of the kits will generally include at least one vial, test tube, flask, bottle, syringe and/or other container into which a first antibody specific for one of the polypeptides or a first nucleic acid specific for one of the polynucleotides of the present invention may be placed and/or suitably aliquoted. Where a second and/or third and/or additional component is provided, the kit will also generally contain a second, third and/or other additional container into which this component may be placed. Alternatively, a container may contain a mixture of more than one antibody or nucleic acid reagent, each reagent specifically binding a different marker in accordance with the present invention. The kits of the present invention will also typically include means for containing the antibody or nucleic acid probes in close confinement for commercial sale. Such containers may include injection and/or blow-molded plastic containers into which the desired vials are retained.

The kits may further comprise positive and negative controls, as well as instructions for the use of kit components contained therein, in accordance with the methods of the present invention.

### 5.3. In Vivo Imaging

The various markers of the invention also provide reagents for in vivo imaging such as, for instance, the imaging of HOXC6 associated with ovarian cancer using labeled reagents that detect (i) nucleic acids associated with particular HOXC6, (ii) a polypeptides associated with a particular HOXC6. In vivo imaging techniques may be used, for example, as guides for surgical resection or to detect the distant spread of ovarian cancer.

For in vivo imaging purposes, reagents that detect the presence of these proteins or genes, such as antibodies, may be labeled with a positron-emitting isotope (e.g., 18F) for positron emission tomography (PET), gamma-ray isotope (e.g., 99mTc) for single photon emission computed tomography (SPECT), a paramagnetic molecule or nanoparticle (e.g., Gd³⁺ chelate or coated magnetite nanoparticle) for magnetic resonance imaging (MRI), a near-infrared fluorophore for near-infra red (near-IR) imaging, a luciferase (firefly, bacterial, or coelenterate), green fluorescent protein, or other luminescent molecule for bioluminescence imaging, or a perfluorocarbon-filled vesicle for ultrasound.

Furthermore, such reagents may include a fluorescent moiety, such as a fluorescent protein, peptide, or fluorescent dye molecule. Common classes of fluorescent dyes include, but are not limited to, xanthenes such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes, polymethine, oxabenzanthrane, xanthene, pyrylium, carbostyl, perylene, acridone, quinacridone, rubrene, anthracene, coronene, phenanthrecene, pyrene, butadiene, stilbene, lanthanide metal chelate complexes, rare-earth metal chelate complexes, and derivatives of such dyes. Fluorescent dyes are discussed, for example, in U.S. Pat. No. 4,452,720 (Harada et al.); U.S. Pat. No. 5,227,487 (Haugland and Whitaker); and U.S. Pat. No. 5,543,295 (Bronstein et al.). Other fluorescent labels suitable for use in the practice of this invention include a fluorescein dye. Typical fluorescein dyes include, but are not limited to, 5-carboxyfluorescein, fluorescein-5-isothiocyanate, and 6-carboxyfluorescein; examples of other fluorescein dyes can be found, for example, in U.S. Pat. No. 4,439,356 (Khanna and Colvin); U.S. Pat. No. 5,066,580 (Lee), U.S. Pat. No. 5,750,409 (Hermann et al.); and U.S. Pat. No. 6,008,379 (Benson et al.). The kits may include a rhodamine dye, such as, for example, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®, and other rhodamine dyes. Other rhodamine dyes can be found, for example, in U.S. Pat. No. 5,936,087 (Benson et al.), U.S. Pat. No. 6,025,505 (Lee et al.); U.S. Pat. No. 6,080,852 (Lee et al.). The kits may include a cyanine dye, such as, for example, Cy3, Cy3B, Cy3.5, Cy5, Cy5.5, Cy7. Phosphorescent compounds including porphyrins, phthalocyanines, polyaromatic compounds such as pyrenes, anthracenes and acenaphthenes, and so forth, may also be used.

### 5.4. Methods to Identify Compounds

A variety of methods may be used to identify compounds that modulate ovarian cancer and prevent or treat ovarian cancer progression. Typically, an assay that provides a readily measured parameter is adapted to be performed in the wells of multi-well plates in order to facilitate the screening of members of a library of test compounds as described herein. Thus, in one embodiment, an appropriate number of cells can be plated into each well of a multi-well plate, and the effect of a test compound on HOXC6 associated with ovarian cancer can be determined. The compounds to be tested can be any small chemical compound, or a macromolecule, such as a protein, sugar, nucleic acid or lipid. Typically, test compounds will be small chemical molecules and peptides. Essentially any chemical compound can be used as a test compound in this aspect of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods are used which involve providing a combinatorial chemical or peptide library containing a large number of potential therapeutic compounds. Such “combinatorial chemical libraries” or “ligand libraries” are then screened in one or more assays, as described herein, to identify those library members (particular chemical species or subclasses) that display a desired characteristic activity. In this instance, such compounds are screened for their ability to modulate the HOXC6 associated with ovarian cancer. A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Unless defined otherwise, all technical and scientific terms used herein have the same meaning as commonly understood by one of ordinary skill in the art to which this invention belongs. The article “a” and “an” are used herein to refer to one or more than one (i.e., to at least one) of the grammatical object(s) of the article. By way of example, “an element” means one or more elements.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The following Examples further illustrate the invention and are not intended to limit the scope of the invention. In particular, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

### 6. EXAMPLES

### 6.1. Example 1

The industry standard indirect ELISA protocol did not provide acceptable results. It gave poor and inconsistent results with blood serum, which was not suitable for diagnostic purposes. Thus the overall protocol was improved and optimized to the following:

In one set of experiments, the wells of a PVC 96 well micro-titer plate (Nunc, Roskilde, Denmark) were coated with 100 ul of the anti HOXC6 capture antibody (Mouse anti human HOXC6 (SC-376330, HOXC6 (B-7), Santa Cruz, Calif.) at a concentration of 1 μg/ml in coating buffer, pH 9.6. The coating buffer consisted of 0.1 M carbonate/bicarbonate buffer pH 9.6 (Sigma-Aldrich, USA). The plates were covered with saran wrap and incubated for 18 h at 4° C. The coating solution was removed by inverting the plate in decanter and the wells were washed twice by adding 360 ul of PBS to each well followed by decanting, done at room temperature (23-25° C.). The phosphate buffer solution pH 7.4 (PBS) consisted of 0.1 M phosphate buffered saline, 0.137 M NaCl and 0.003 M KCl (Sigma-Aldridge, USA). The remaining protein sites were blocked by adding 200 ul of blocking buffer to each well. The blocking buffer solution consisted of a PBS solution (PH 7.4) with added 1% (w/v) bovine serum albumin (BSA) purchased from Sigma (Sigma Aldridge, USA). Plates were covered with saran wrap and incubated for 2 h at room temperature (23-25° C.). After washing twice by adding 360 ul of PBS to each well followed by decanting, done at room temperature (23-25° C.), 100 ul of 1 to 100 dilution of patient's serum diluted in binding buffer (PBS solution with added 1% (W/V) BSA, pH 7.4) was added to the appropriate wells and incubated for 2 h at room temperature (23-25° C.). The serum was prepared previously from Human subjects blood collected in a serum separating tube (The BD Vacutainer® SST™, BD Technologies, USA), spun at room temperature (23-25° C.), at a speed of 1200 RCF for 10 minutes in a swinging bucket centrifuge (Beckman Coulter, Inc., USA). The serum portion was collected, divided into 25 ul aliquots into 0.5 ml microfuge tubes and stored at −80° C. freezer. At this step, HOXC6 partial Recombinant Protein (H00003223-Q01, Novus Biological, LLC, Littleton, Colo., USA) in a concentration of 1 μg/ml in binding buffer was used to create the standard curve for HOXC6 protein. Plates were washed four times by adding 360 ul of PBS to each well followed by decanting, done at room temperature (23-25° C.), and 100 ul of 1 to 100 dilution of goat anti human HOXC6 (SC-46135, Santa Cruz Bio., CA, USA) detection primary antibody (200 ug/ml) diluted in binding buffer was added, covered by Saran wrap and incubated at room temperature (23-25° C.) for 2 hrs. Plates were then washed four times by adding 360 ul of PBS to each well followed by decanting, done at room temperature (23-25° C.), and 100 ul of 1 to 100 dilution of Bovine anti-goat IgG-HRP (SC-2350, Santa Cruz Bio, USA) secondary antibody (400 ug/ml) diluted in binding buffer was added and incubated for 2 h at room temperature (23-25° C.). The plates were washed 4 times by adding 360 ul of PBS to each well followed by decanting, done at room temperature (23-25° C.), and 100 ul of TMB enzyme substrate (3,3′,5,5′-tetramethylbenzidine, TMB enzyme substrate kit, Part#34021, Thermo Scientific, USA) was added to each well, and the plates were incubated for 5 min at room temperature (23-25° C.). Color development was stopped by addition of 50 ul of 2 M sulfuric acid. The optical density was read at 450 nm with a micro plate reader (MultiSkan Go, Thermo Scientific, U.S.A.) within 15 min after stopping the reaction and a copy of printed data was obtained.

Tissue and Serum Collection: Ovarian tissue specimens were obtained during surgery from human subjects with ovarian cancer or other gynecologic conditions according to an IRB-approved protocol at Carolinas Medical Center. Tissue samples were placed in a standard sized cryomold (Sakura Finetek USA, Inc., Torrance, Calif.), covered with Optimal Cutting Temperature (OCT) compound (Sakura Finetek USA, Inc., Torrance, Calif.), frozen and stored at −80° C. until used. Matched blood serum samples were collected by using a BD Vacutainer® SST™ serum separating tube (BD Biosciences, San Jose, Calif.) according to the manufacturer's instructions, and stored at −80° C. for later use.

Laser Capture Micro-dissection (LCM): OCT embedded samples were serially sectioned into 8 μm sections using Fisherbrand Superfrost® */Plus Microscope slides (Fisher Scientific, Pittsburgh, Pa.) by a Leica CM 1850 UV Cryostat (Leica Microsystems Inc., Bannockburn, Ill.). The sections were prepared for LCM, using the HistoGene LCM Frozen Section Staining kit (Applied Biosystems, Life Technologies, Co., Carlsbad, Calif.) according to the manufacturer's instructions. After staining, samples were immediately micro-dissected by an Arcturus® PixCell® IIe LCM (Molecular Devices, LLC Sunnyvale, Calif. Normal epithelium and tumor cells were separately collected from appropriate sections. The residual slide material was used to determine RNA quality [31].

RNA preparation: RNA extraction of microdisected-captured cells, and the residual slide material were performed using a PicoPure RNA Isolation kit (Applied Biosystems, LifeTechnologies, Co., Carlsbad, Calif.) according to the manufacturer's protocols. The RNA integrity was measured using an Agilent 2100 Bioanalyzer (Agilent Technologies, Inc., Santa Clara, Calif.) as described by the manufacturer [31].

cDNA Synthesis and amplification: Complimentary DNA (cDNA) generation and amplification was performed using the Whole Transcriptome WT-Ovation Pico RNA Amplification System Kit (NuGEN Technologies Inc, San Carlos, Calif.), following the manufacturer's recommendation. The cDNA products were quantified using a Nanodrop 1000 spectrophotometer (NanoDrop Products, Wilmington, Del.) and was used for microarray sample preparation, and qPCR confirmation assays.

Exon Microarray sample preparation and Hybridization: Approximately 3 μg of SPIA amplified cDNA was used to continue to ST-cDNA conversion using the WT-Ovation Exon Module (NuGEN Technologies Inc, San Carlos, Calif.). 5 μg ST-cDNA was fragmented and labeled with FL-Ovation cDNA Biotin Module V2 kit (NuGEN Technologies Inc, San Carlos, Calif.), then hybridized using Affymetrix Human Exon 1.0 ST arrays (Affymetrix, Inc., Santa Clara, Calif.). The microarray hybridization procedure was performed using a GeneChip Hybridization Oven 640, GeneChip Fluidics Station 450, and GeneChip Scanner 3000 7G with Autoloader (Affymetrix, Inc., Santa Clara, Calif.). For the In Silico Quality Control, each array was required to pass preliminary quality control including assessment of spike-in controls and the total distribution of intensities compared to manufacturer's criteria [31].

Quantitative RT-PCR: Total RNA was isolated from 106-107 ovarian cancer cell line SKOV3 (American Type Culture Collection, Manassas, Va.), and normal ovarian surface epithelial cell line, HOSE [32] using Trizol reagent (Invitrogen Co, Carlsbad Calif.). The extracted RNA was purified using RNeasy Mini Kit (Qiagen, Inc, Valencia, Calif.). 50 ng of the RNA was reverse transcribed to cDNA in 20 ul total volume using QuantiTect Reverse Transcription Kit and protocol (Qiagen Inc, Valencia, Calif.). Approximately 50 ng of cDNA were then amplified using 500 nM of Prime Time qPCR primers for HOXC6 (Hs.PT.51.3113294) and GAPDH (Hs.PT.51.2918858.g), (Integrated DNA Technologies Inc., San Jose, Calif.). GAPDH was used to normalize expression data. SKOV3 and HOSE cell lines were used as positive and negative reference samples. Each primer was researched to ensure that the same transcripts would be detected by real-time PCR as were detected by microarray analysis. Analysis of data was carried out according to the Comparative Ct method for relative quantitation of gene expression [33]. Real-time quantitative PCR was carried out using the Applied Biosystems® 7500 Fast Real-Time PCR System (Applied Biosystem, Carlsbad, Calif.), and the QuantiTect® SYBR Green PCR kit (Qiagen, Inc, Valencia, Calif.) according to the manufactures instructions. The ABI 7500 Fast instrument was operated under the following thermal cycling conditions: Initial heat activation of 95° C. for 15 min, followed by 40 cycles of denaturation of 94° C. for 15 s, annealing of 60° C. for 30 s, and extension of 72° C. for 30 s. The PCR products were checked using ethidium bromide-stained 2% agarose gels. The standard and unknown samples were assayed in triplicate.

Indirect Sandwich Enzyme-Linked Immuno-sorbent Assay: The developed adopted general procedure is summarized as follow: The wells of a micro-titer plate (Nunc, Roskilde, Denmark) were coated with 100 ul of the anti HOXC6 capture antibody (B-7; Santa Cruz Biotechnology, Inc., CA) at a concentration of 1 μg/ml in coating buffer, pH 9.6, consisted of 0.1 M carbonate/bicarbonate buffer (Sigma-Aldrich, St. Louis, Mo.). The plates were incubated overnight at 4° C. followed by washing twice with PBS (Sigma-Aldridge, St. Louis) and were blocked with 200 ul of blocking buffer consisted of a PBS solution with added 1% (w/v) bovine serum albumin (BSA). The plates were incubated for 2 h at room temperature followed by washing with PBS twice. A 1:100 dilution of patient's serum was added to the appropriate wells and incubated for 2 h at room temperature. The HOXC6 partial Recombinant Protein (Novus Biological, LLC, Littleton, Colo.) in a concentration of 1 μg/ml was used to create the standard curve for HOXC6 protein. Plates were washed, and a 1:100 dilution of goat anti human HOXC6 (N-13; Santa-Cruz Biotechnology, Inc., Santa Cruz, Calif.) detection primary antibody (200 ug/ml) was added, and incubated at room temperature for 2 h. Plates were then washed four times and a 1:100 dilution of Bovine anti-goat IgG-HRP (Santa Cruz Biological, Inc. Santa Cruz, Calif.) secondary antibody (400 ug/ml) was added and incubated for 2 h at room temperature. The plates were washed 4 times and 100 ul of 3,3′,5,5′-tetramethylbenzidine (TMB) enzyme substrate (Thermo Scientific Inc, Barrington, Ill.) was added to each well, followed by incubation at room temperature. Color development was stopped by addition of 2 M sulfuric acid. The optical density was read at 455 nm with a micro plate reader (MultiSkan Go) (Thermo Scientific, Inc., Barrington, Ill.) according to manufacturer's recommendation [34].

Immunohistochemistry: Immunohistochemistry (IHC) was performed to evaluate presence of HOXC6 target protein in tissue sections. OCT embedded samples were cut into 8 μm sections. The OCT material was removed by soaking the slides in deionized water then fixed with 2% paraformaldehyde. The sections were incubated in 3% H2O2 for 5 min at room temperature. Antigen retrieval was carried out in citrate buffer for 20 min in a humidified chamber. Tissue sections were washed with PBS and incubated 45 min with 50% fetal bovine serum (FBS) blocking solution. After removal of blocking solution, sections were incubated overnight at 4° C. in a 1:250 dilution of goat-anti-human HOXC6 antibody (Santa-Cruz Biotechnology Inc., Santa Cruz, Calif.). Following wash in 1×PBS, sections were incubated with a 1:250 dilution of biotinylated rabbit anti-goat secondary antibody for 45 min at room temperature (R&D systems Inc., Minneapolis, Minn.). A streptavidin-enzyme conjugate (BD Biosciences, San Jose, Calif.) was added to the slides according to the manufacturer instructions. Specific signals were visualized by incubation with streptavidin HRP followed by diaminobenzidine (DAB) as a chromogen (BD Biosciences, San Jose, Calif.). Counterstaining was performed with Mayer's hematoxylin (Sigma-Aldrich, St. Louis, Mo.), and slides were covered with Permount (Fisher Scientific, Pittsburgh, Pa.). Images were captured by Kodak imaging system and stored digitally for analysis. A section of placenta tissue known to express HOXC6 protein was used as a positive control. Negative controls were prepared by secondary antibody only (data not shown). Additional sections were stained with hematoxylin (Richard-Allen Scientific, KALAMAZOO, Mich.) and eosin (Sigma-Aldrich, St. Louis, Mo.) (H&E) for comparison using standard protocols as suggested by manufacturer.

### 3.1. Data Analysis

Transcriptional microarray analysis was performed on eight serous ovarian carcinoma tissue samples and three normal ovarian tissue samples using the Affymetrix Human Exon 1.0 ST arrays (Affymetrix, Inc., Santa Clara, Calif.). These samples were categorized into normal and malignant serous ovarian origins according to pathological diagnosis. Rather than use of bulk tissue samples for study, use of laser-capture microdissection (LCM) was used to ensure specific collection of target tumor cells from malignant tissues (TM) or normal epithelium cells from non-tumorous ovarian tissue (NNE) of human subjects and thus increase sample purity and reliability of results. All study subjects were postmenopausal women at the time of surgery. The mean age at time of collection was 64 years for non-malignant samples and 65 years for malignant tumor samples.

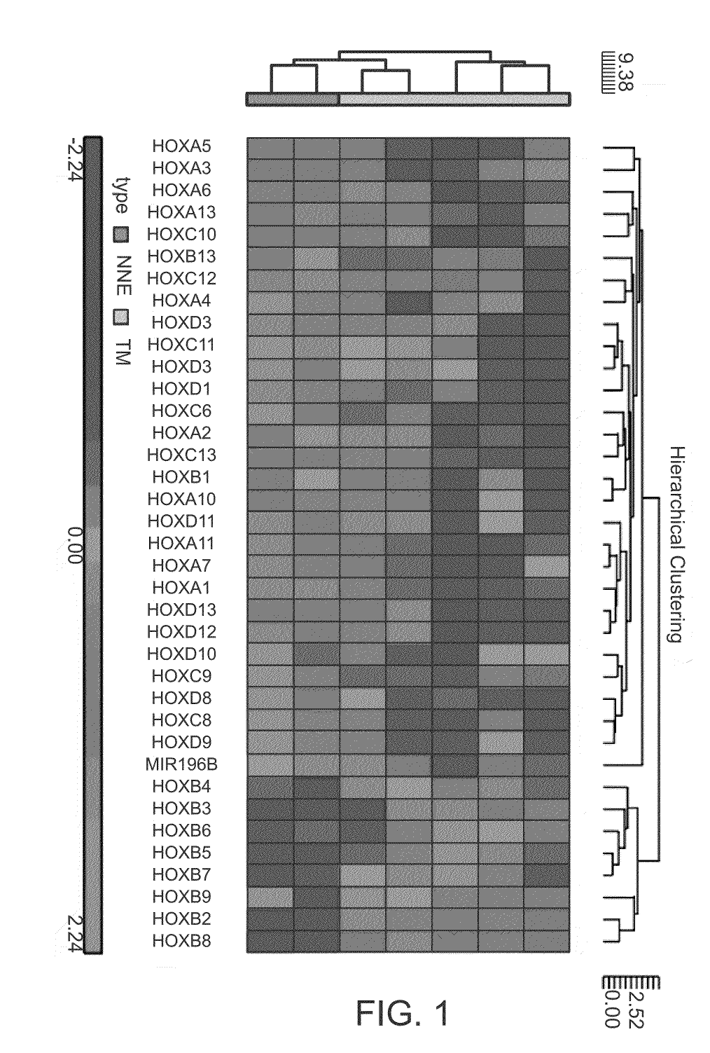

Expression analysis was performed by Partek Genomics Suite 6.12.0530 using 1-way ANOVA model by Method of Moments [35]. The Fisher's Least Significant Difference (LSD) contrast (s) method [36] was performed to compare expression of the normal ovarian tissue samples to the serous malignant tissue samples. A total of 351 transcripts demonstrated significantly altered expression (data not shown). Based on our previous ovarian cancer gene expression studies showing significant patterns of HOX gene up- and down-regulation [28], we used this sample set to determine HOX gene expression patterns from the existing 37 HOX gene probe sets in Affymetrix Human Exon 1.0 ST array (FIG. 1). We determined the fold-change expression between serous malignant and normal ovarian tissues of selected genes from the HOX family (Table 1). The most significantly up-regulated genes include HOXB2 and HOXB3, and the most significantly down-regulated gene includes HOXC6. We noted that several previously identified dysregulated HOX genes were not altered (HOXA7, A10, D1; p-values>0.05), likely due to the difference between bulk tissue extraction used previously and selective LCM dissection of tumor cells used here.

TABLE 1
Up- and down-regulated selected HOX genes
dysregulated in serous ovarian carcinomas.

| Gene Name | Gene ID | Fold Change | p-value |
|---|---|---|---|
| Upregulated: | | | |
| HOXA7 | NM_006896 | +1.05 | 0.82399 |
| HOXA10 | NM_018951 | +1.08 | 0.70020 |
| HOXB2 | NM_002145 | +2.28 | 0.00248 |
| HOXB3 | NM_002146 | +2.49 | 0.00209 |
| HOXB5 | NM_002147 | +1.85 | 0.02028 |
| HOXB7 | NM_004502 | +1.50 | 0.01558 |
| HOXD1 | NM_024501 | +1.21 | 0.47472 |
| Downregulated: | | | |
| HOXC6 | NM_004503 | −2.12 | 0.00214 |

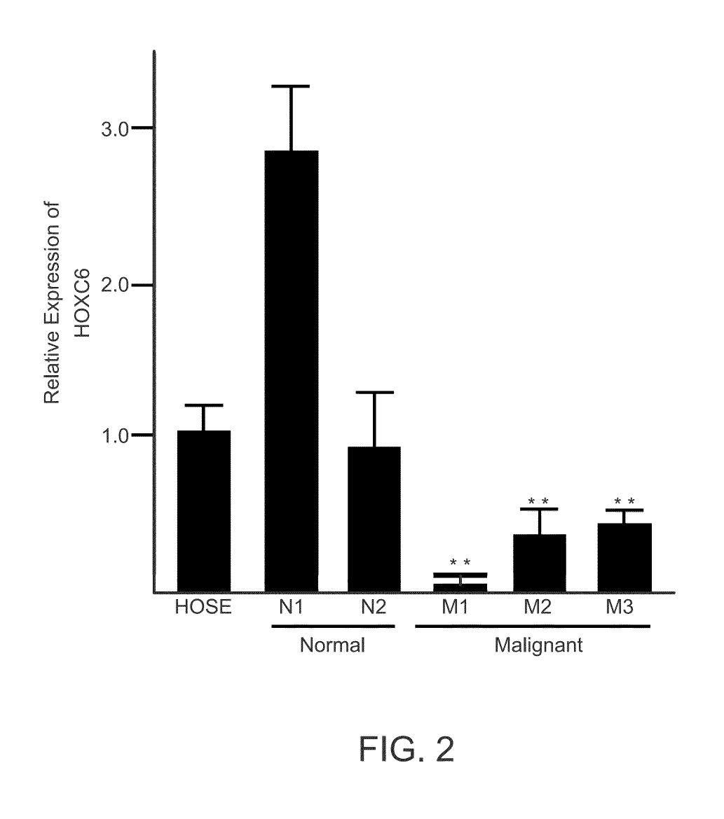

In order to validate microarray data, quantification of gene expression was carried out using quantitative RT-PCR. qRT-PCR quantification of HOXC6 mRNA was consistent with the microarray data, with malignant ovarian samples showing lower levels of HOXC6 mRNA than normal ovarian tissue samples. HOXC6 exhibited a 7-fold increase in expression in normal ovary compared to serous ovarian cancer, (Table 1 and FIG. 2).

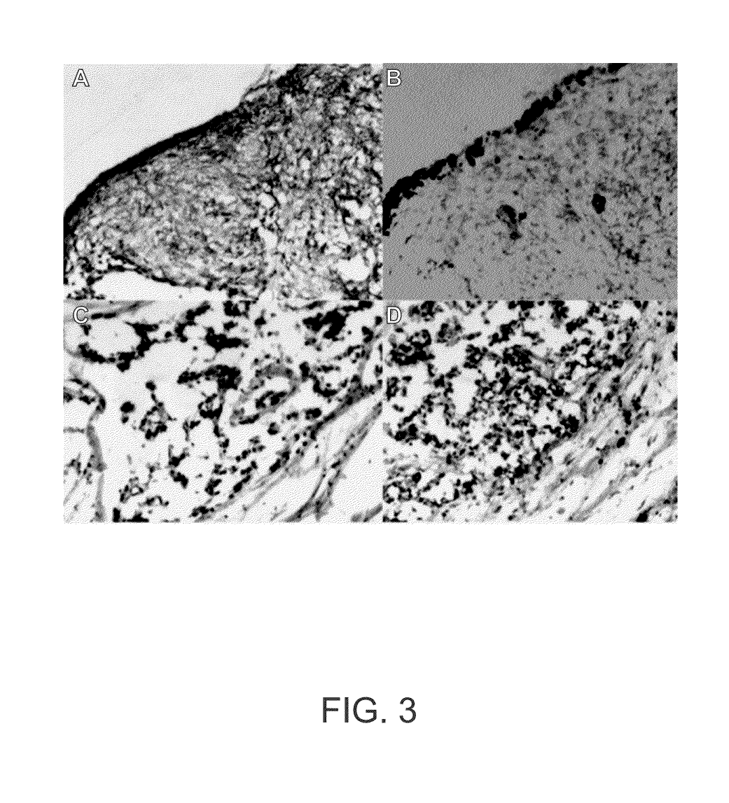

Immunohistochemistry allowed visualization of the protein product for HOXC6 in ovarian tissue samples. To date, normal ovarian surface epithelium has not been shown to express HOX genes to any measurable degree. HOXC6 analysis indicated that the elevated levels of mRNA in normal ovarian tissue samples is translated into protein, and that these proteins are translocated to the nucleus. FIG. 3 Panel B represents HOXC6 protein localization in normal ovary. The normal ovarian surface epithelial layer stains positively for HOXC6 with absent staining in the underlying stroma. By contrast, HOXC6 staining is absent in the malignant ovarian tissue shown in FIG. 3 Panel D.

An indirect sandwich enzyme-linked immuno-sorbent assay (ELISA) was performed to detect HOXC6 protein in blood serum of eight malignant and three normal human subjects under-going surgery. In addition, ELISA was performed to detect HOXC6 protein in serum of seven normal female volunteers with ovaries without any known disease or condition. A serial dilution of known amount of commercially available HOXC6 partial Recombinant Protein was used to create the standard curve for HOXC6 protein as well as validation sample. The HRP-TMB enzyme substrate reaction was detected by using a microplate reader at wavelength of 455 nm. A fold change analysis was performed by computing the ratio of the mean of OD reading values of the eight malignant samples compared to the average mean of OD reading values of eight normal samples including the volunteers. An average of 1.3-fold (range of 1.1-1.6) decreased amounts of HOXC6 was detected in malignant serum samples as compared to normal serum samples (data not shown). These findings support those seen in the microarray, RT-PCR and IHC HOXC6 analysis.

Research has shown altered expression levels of many HOX genes in several cancers, including endometrial, cervical, pancreatic, thyroid, and lung [6-11]. Altered expression of HOX genes has also been demonstrated in ovarian cancer [28-30], although little has been known about HOXC6 and ovarian cancer. The connection of HOX genes to oncogenic pathways such as the RAS signaling cascade and angiogenic pathways involving vascular endothelial growth factor (VEGF) and basic fibroblast growth factor (bFGF) has been established [40-42]. HOX genes are also involved in the activation of T lymphocytes that have been demonstrated to be responsible for the immune response to ovarian carcinoma [45, 46]. These factors make the HOX genes of multifaceted interest in the pathogenesis of ovarian cancer.

During development, HOX genes within each of the 4 clusters are expressed in sequence along the chromosome as the spatial arrangement of the developing tissue progresses from anterior to posterior. This is termed segmental polarity, and is seen in the mammalian female paramesonephric duct as it develops into the fallopian tube, uterus, cervix and upper vagina [29]. These structures develop according to the Müllerian pathway of differentiation and are characterized by highly structured epithelial architecture. HOX gene expression remains active in these tissues throughout adulthood and is thought to function in the maintenance of cell identity and the highly differentiated phenotype [46]. The ovary is not a part of this Müllerian system of development, and the normal ovarian surface epithelium maintains a highly undifferentiated structure. To date, normal ovarian surface epithelium has not been shown to express HOX genes to any measurable degree; the finding of HOXC6 increased expression shown here (FIG. 3) is a novel finding. Conversely, HOXC6 is down-regulated in epithelial ovarian cancer (FIG. 3). This down-regulation of HOXC6 in ovarian carcinoma seems to be unique in the HOX family of genes as multiple HOX genes are known to be up-regulated including HOXA7, HOXB2, and HOXB7 [47, 48].

The HOXC genes are involved in transcriptional activation during embryonic development. The HOXC6 homebox is found in 3 different mRNAs and transcripts coexist in several cells and tissues to include fibroblasts, spinal cord, limbs and skin [23]. Overexpression of HOXC6 has been demonstrated in the human malignancies meduloblastomas, osteosarcomas, breast, lung, gastrointestinal, head and neck squamous, and prostate carcinomas, leukemia as well as normal trophoblast [4, 15, 24-27]. Although not previously reported in human ovary, increased expression of HOXC6 occurs in murine ovarian tissue [14]. HOXC6 function may be modulated by a variety of secreted factors such as growth factors, cytokines, and hormones. Evidence that HOXC6 is regulated by TGF-beta suggests HOX genes are targets for the TGF-beta superfamily of genes and serve as a mechanism for growth factors to exert their effect on development and carcinogenesis [18]. Estradiol regulation of HOXC6 has specific implications to HOXC6 function in ovarian and Müllerian tissues [19, 20]. Although HOXC6 is over-expressed in hormone responsive breast and prostate tumors [23, 50], it is notable that the observed elevated expression of HOXC6 in mammary glands of ovariectomized female mice suggests negative regulation of HOXC6 by ovarian hormones [51, 52]. This finding supports the idea that HOXC6 expression is potentially both positively and negatively regulated by steroid hormones in a tissue-dependent manner. Hypermethylation in a number of tumors leads to loss of tumor suppressor function in multiple tumor types. Transcription of HOXC6 is repressed by hypermethylation via Lsh protein mediated Polymerase II stalling [53] and in association with MLL1 and MLL4 histone methyltransferase binding to the HOXC6 promoter region [19]. Elevated methylation associated with decreased gene expression has been observed in cases of high grade serous ovarian cancer [54] although the specific status of HOXC6 promoter region has not been examined.

The inventors finding of HOXC6 up-regulation on normal ovarian epithelium and down-regulation in serous ovarian carcinoma, the most common ovarian malignancy, suggests a role as a biologic marker for ovarian cancer. In a clinical oncology setting, biologic markers can have various functions including detection, monitoring response to treatment, or serving as therapeutic target. For example, identification of biologic markers useful for early detection of ovarian cancer has been disappointing. Single markers like CA125 have not met the statistical bar as a screening test. Clarke-Pearson DL: Clinical practice. Screening for ovarian cancer. N Engl J Med 2009, 361(2):170-177. Attempts at developing assays with multiple biomarkers have been equally unsuccessful. One study of a tumor-marker panel consisting of CA125, leptin, prolactin, osteopontin, insulin-like growth factor II, and macrophage migration inhibitory factor improved screening sensitivity and specificity but still fell short of the acceptable statistical bar. Visintin I, Feng Z, Longton G, Ward D C, Alvero A B, Lai Y, Tenthorey J, Leiser A, Flores-Saaib R, Yu H et al: Diagnostic markers for early detection of ovarian cancer. Clin Cancer Res 2008, 14(4):1065-1072. These multiple marker panels are designed to detect elevations in the selected proteins. This unique loss of HOXC6 expression in ovarian carcinoma may have value in the future development of tumor-marker panels enhancing sensitivity and specificity. The finding of HOXC6 down-regulation offers a change in the paradigm in that down-regulation of a gene specific to a malignancy may be important clinically in the context of detection as well as biologically in the process of oncogenesis.

### 6.2. Example 2

### Tissue and Serum Collection

Ovarian tissue specimens were obtained during surgery from patients with ovarian cancer or other gynecologic conditions according to an IRB-approved protocol at Carolinas Medical Center. All available patient data is presented in Table 2. All patients with serous carcinoma were stage III and IV, grade 3 and received platinum and taxane based chemotherapy after surgery. Tissue samples were placed in a standard sized Cryomold®cryomold (Sakura Finetek USA, Inc., Torrance, Calif.), covered with Optimal Cutting Temperature (OCT) compound (Sakura Finetek USA, Inc., Torrance, Calif.), frozen and stored at −80° C. Originally seven malignant matched and 11 non-malignant blood serum samples were collected by using a BD Vacutainer® SST™ serum separating tube (BD Biosciences, San Jose, Calif.) according to the manufacturer's protocols, and stored at −80° C. To further validate the ELISA findings, 32 additional other serum samples were collected from pre- and postmenopausal women without ovarian cancer, and serum from additional 14 patients with stage III and IV, grade 3 serous carcinoma of the ovary or fallopian tube—total of 21 malignant and 43 non-normal serum samples.

Laser Capture Micro-Dissection (LCM)

OCT embedded samples were serially sectioned into 8 m sections using Fisherbrand Superfrost® */Plus Microscope slides (Fisher Scientific, Pittsburgh, Pa.) by a Leica CM 1850 UV Cryostat (Leica Microsystems Inc., Bannockburn, Ill.). Sections were prepared for LCM using Histogene® LCM Frozen Section Staining kit (Applied Biosystems, Life Technologies, Co., Carlsbad, Calif.) according to the manufacturer's protocols. After staining, samples were immediately micro-dissected by an Arcturus® PixCell® IIe LCM (Molecular Devices, LLC, Sunnyvale, Calif.). Normal epithelium and tumor cells were separately collected from appropriate sections. The residual slide material was used to determine RNA quality [31].

RNA Preparation

RNA extraction of LCM captured cells, and the residual slide materials were performed using a PicoPure® RNA Isolation kit (Applied Biosystems, LifeTechnologies, Co., Carlsbad, Calif.) according to manufacturer's protocols. RNA integrity was measured using an Agilent 2100 Bioanalyzer (Agilent Technologies, Inc., Santa Clara, Calif.) as described by the manufacturer [31].

cDNA Synthesis and Amplification

Complimentary DNA (cDNA) generation and amplification was performed using the Whole Transcriptome WT-Ovation™ Pico RNA Amplification System Kit (NuGEN Technologies Inc., San Carlos, Calif.), according to manufacturer's protocols. cDNA was quantified using a NanoDrop 1000 spectrophotometer (NanoDrop Products, Wilmington, Del.) and used for microarray sample preparation and qPCR confirmation assays.

Exon Microarray Sample Preparation and Hybridization

Approximately 3 μg of single primer isothermal amplified (SPIA) cDNA was used to continue to sense-strand cDNA (ST-cDNA) conversion using the WT-Ovation™ Exon Module (NuGEN Technologies Inc., San Carlos, Calif.). 5 μg ST-cDNA was fragmented and labeled with FL-Ovation™ cDNA Biotin Module V2 kit (NuGEN Technologies Inc., San Carlos, Calif.) then hybridized using GeneChip® Affymetrix Human Exon 1.0 ST arrays (Affymetrix, Inc., Santa Clara, Calif.). Microarray hybridization was performed using a GeneChip® Hybridization Oven 640, GeneChip® Fluidics Station 450, and GeneChip® Scanner 3000 7G with Autoloader (Affymetrix, Inc., Santa Clara, Calif.). For the In Silico Quality Control, each array passed preliminary quality control including assessment of spike-in controls and the total distribution of intensities compared to manufacturer's criteria [31].

Quantitative RT-PCR

RNA from primary ovarian tissue samples was prepared as described above. Normal ovarian surface epithelial cell line HOSE 6-3 [32] was used as reference sample. Ovarian cancer cell lines SKOV-3 and Caov-3, and other selected cell lines such as SW626, MDA-MB-231 [Breast], and HeLa [Cervix] (American Type Culture Collection, Manassas, Va.) were used as cell culture model for this study. Total RNA was isolated from 10⁶-10⁷ cells using TRIzol® Reagent (Invitrogen Co, Carlsbad Calif.). Extracted RNA was purified using RNeasy Mini Kit (Qiagen, Inc., Valencia, Calif.). 50 ng RNA was reverse transcribed to cDNA in 20 total volume using QuantiTect Reverse Transcription Kit (Qiagen Inc., Valencia, Calif.). 50 ng cDNA was amplified using 500 nm of PrimeTime® qPCR primers for HOXC6 [Hs.PT.51.3113294)] and GAPDH [Hs.PT.51.2918858.g] (Integrated DNA Technologies Inc., San Jose, Calif.). GAPDH was used to normalize expression data. Primers were selected to amplify by real-time PCR the same transcripts as detected by microarray. Analysis of data was carried out according to the Comparative Ct method for relative quantitation of gene expression [33]. Real-time quantitative PCR was carried out using the Applied Biosystems® 7500 Fast (ABI 7500 Fast) Real-Time PCR System (Applied Biosystem, Carlsbad, Calif.), and the QuantiTect® SYBR Green PCR kit (Qiagen, Inc., Valencia, Calif.) according to the manufacturer's protocols. The ABI 7500 Fast instrument was operated under the following thermal cycling conditions: Initial heat activation of 95° C. for 15 min, followed by 40 cycles of denaturation of 94° C. for 15 s, annealing of 60° C. for 30 s, and extension of 72° C. for 30 s. The PCR products were checked using ethidium bromide-stained 2% agarose gels. Both standard and primary tissue samples were assayed in triplicate.

Immunohistochemistry

Tissue sections were incubated in 3% H2O2 for 5 min at room temperature and washed with PBS, then incubated for 45 min with 50% fetal bovine serum blocking solution. After removal of blocking solution, sections were incubated overnight at 4° C. in a 1:250 dilution of goat-anti-human HOXC6 antibody (Santa-Cruz Biotechnology Inc., Santa Cruz, Calif.). Sections were washed and incubated with a 1:250 dilution of biotinylated rabbit anti-goat secondary antibody for 45 min at room temperature (R&D systems Inc., Minneapolis, Minn.). A streptavidin-enzyme conjugate (BD Biosciences, San Jose, Calif.) was added to the slides according to the manufacturer's protocols. Specific signals were visualized by incubation with streptavidin HRP followed with adding diaminobenzidine (DAB) as a chromogen (BD Biosciences, San Jose, Calif.). Counterstaining was performed with Mayer's hematoxylin (Sigma-Aldrige, St. Louis, Mo.). Images were captured and stored digitally for analysis.

Indirect Sandwich Enzyme-Linked Immunosorbent Assay (ELISA)

The developed and optimized protocol is summarized as follows: Wells of a micro-titer plate (Nunc, Roskilde, Denmark) were coated with 100 μL of anti-HOXC6 capture antibody (B-7; Santa Cruz Biotechnology, Inc., CA) at 1 μg/mL in coating buffer (pH 9.6) of 0.1 M carbonate/bicarbonate buffer (Sigma-Aldrich, St. Louis, Mo.). The plates were incubated overnight at 4° C. followed by washing 2× with PBS (Sigma-Aldridge, St. Louis) and were blocked with 200 μL of blocking buffer of PBS −1% (w/v) bovine serum albumin (BSA). The plates were incubated 2 h at room temperature followed by washing 2× with PBS. A 1:100 dilution of patient's serum was added and incubated for 2 h at room temperature. To create the standard curve for HOXC6 protein, the HOXC6 partial recombinant protein (Novus Biological, LLC, Littleton, Colo.) at 1 μg/mL was used. Plates were washed, and a 1:100 dilution of goat anti-human HOXC6 primary antibody (N-13; Santa-Cruz Biotechnology, Inc., Santa Cruz, Calif.) at 200 μg/mL was added, and incubated at room temperature for 2 h. Plates were washed 4× and a 1:1000 dilution of bovine anti-goat IgG-HRP secondary antibody (Santa Cruz Biological, Inc. Santa Cruz, Calif.) at 400 μg/mL was added and incubated for 2 h at room temperature. The plates were washed 4× and 100 μL of 3,3′,5,5′-tetramethylbenzidine (TMB) enzyme substrate (Thermo Scientific Inc., Barrington, Ill.) added to each well, followed by incubation at room temperature. Color development was stopped by addition of 2 M sulfuric acid (Sigma-Aldridge, St. Louis). Optical density was read at 450 nm using Multiskan™ GO Microplate Spectrophotometer (Thermo Scientific, Inc., Barrington, Ill.) according to manufacturer's protocols [34]. Based on the standard curve, the serum samples were analyzed in triplicate.

### Results

In an initial transcriptional microarray screen of 65 ovarian cancer tissue samples, significant patterns of HOX gene dysregulation were observed, and HOXC6 was significantly down regulated [28]. To further characterize the expression of the HOXC6 gene in serous ovarian cancer, transcriptional microarray analysis was performed on seven serous ovarian carcinoma tissue samples and three normal ovarian surface epithelium samples. Use of laser-capture microdissection (LCM) in this study ensured specific collection of target tumor cells from malignant ovarian tissues (MT) or normal epithelium cells from non-malignant ovarian tissues (NE) and thus increased sample purity. Samples were categorized into normal and malignant serous ovarian origins according to pathological diagnosis. All study subjects were postmenopausal women at the time of surgery with mean age of 66 (Table 1). The age groups for non-malignant samples at the time of collection were ⅓<50 and ⅔>50 (data not shown).

Expression analysis was performed by Partek® Genomics Suite™ 6.12.0531 using 1-way ANOVA model by Method of Moments [35]. Fisher's Least Significant Difference contrast(s) method [36] was performed to compare samples. Based on our previous ovarian cancer gene expression study showing significant patterns of HOX gene dysregulation [28], we used this sample set to determine HOX gene expression patterns of the existing 37 HOX gene probe sets in Affymetrix Human Exon 1.0 ST array. We determined the fold-change expression between serous malignant and normal ovarian surface epithelium samples of selected genes from the HOX family (Table 3). The most significantly up-regulated genes include HOXB2 and HOXB3, and the most significantly down regulated gene is HOXC6. We noted that several previously identified dysregulated HOX genes were not significantly altered (HOXA7, HOXA10, HOXD1; p-values>0.05), likely due to the difference between bulk tissue extraction used previously and selective LCM dissection of tumor cells used here.

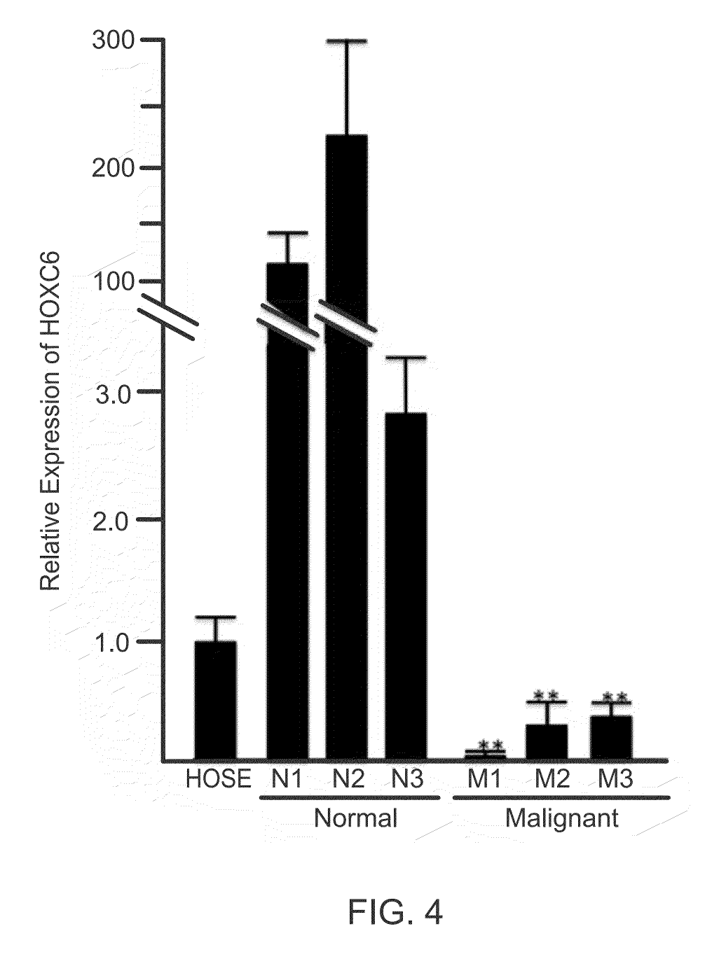

To validate transcriptional microarray data, qRT-PCR was used. HOXC6 exhibited a significant decrease in expression in malignant serous ovarian cancer compared to normal ovarian surface epithelium (FIG. 4). The number of samples tested for qRT-PCR was limited by the availability of cDNA from micro-dissected samples used for microarray.

To confirm in vitro down-regulation of HOXC6 in ovarian cancer tissue samples, 2 ovarian cancer cell lines SKOV-3 and Caov-3 were analyzed by qRT-PCR. In addition, 3 cell lines, MDA-MB-231 (breast), HeLa (cervix), and SW262 (colon metastasized to ovary [39]) were used to determine the specificity of HOXC6 in regard to ovarian cancer. HOXC6 was down-regulated in both ovarian cell lines as well as SW262 (Table 4). However, HOXC6 was highly expressed in both breast and cervical cell lines (Table 4). The variations in HOXC6 expression between the ovarian cell lines could be due to the invasive nature of SKOV-3 over other ovarian cell line. The aggressiveness of SKOV-3 could be due to low and high production of Inhibin A and Activin, respectively [37], and activation of oncogenes such as nm23 and c-erbB-2 [38].

To determine if decreased HOXC6 RNA levels correlate with decreased protein levels, immunohistochemistry (IHC) was performed on a subset of the serous ovarian cancer samples (n=6) and nonmalignant ovarian tissue samples (n=8). IHC demonstrated that HOXC6 protein is present at high levels in normal ovarian surface epithelial cells, normally translocated to the nucleus (FIG. 3). The normal ovarian surface epithelial layer stains positively for HOXC6 with absent staining in the underlying stroma. By contrast, HOXC6 staining is absent in the malignant ovarian tissue likely due to the disordered tissue architecture characteristic of the tumors (FIG. 3). This finding was consistent with the hypothesis that HOXC6 protein is reduced in serous ovarian tumors and supported subsequent study for HOXC6 detection in serum.

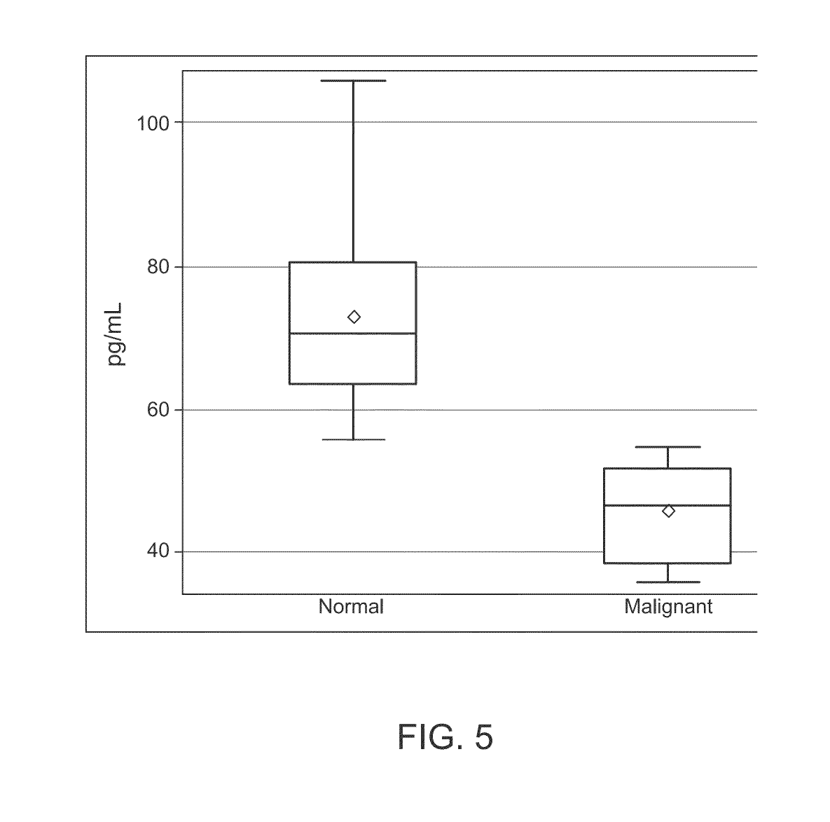

An indirect sandwich enzyme-linked immunosorbent assay (ELISA) was performed to detect HOXC6 protein in serum of 21 women with ovarian cancer and 43 women with ovaries, who did not have diagnosis of ovarian cancer. The cohort of the 64 serum samples included 10 samples matched to the microarray samples (7 malignant and 3 nonmalignant) and additional 14 serum samples from women with ovarian cancer and 40 serum samples from women without ovarian cancer. Serial dilutions of commercially available HOXC6 recombinant protein were used to determine the standard curve as well as control for inter-sample and inter-plate validations. The calculated mean concentration of non-malignant samples was 72.9 pg/mL+23.9/−17.1 pg/mL (stdev=0.122) corresponding to 2.71 pM (235 amino acids, 26.9 kD). The calculated mean concentration of malignant samples was decreased to 45.7 pg/mL+8.78/−9.9 pg/mL (stdev=0.067) corresponding to 1.70 pM (FIG. 5). Consistent with all the other data, the decrease detected in serum samples was statistically significant (p-value=1.18×10⁻¹³).

### Discussion

Studies have shown altered expression levels of HOX genes in multiple cancers, including endometrial, cervical, pancreatic, thyroid, and lung [6-11]. Altered expression of HOX genes has been demonstrated in ovarian cancer [28-30] although little has been known about the possible role of HOXC6 and ovarian cancer pathogenesis. In addition to our previous study [28], we now report a more focused sample set that demonstrates HOXC6 is both significantly down-regulated in serous ovarian cancer tissue and present at significantly reduced levels in serum of patients.

The connection of HOX genes to oncogenic pathways such as RAS signaling cascade and angiogenic pathways involving vascular endothelial growth factor (VEGF) and basic fibroblastic growth factor (bFGF) has been described [40,41,42]. The angiogenic pathways, in particular, are important in the pathogenesis of ovarian cancer and have been developed as specific therapeutic targets for the treatment of ovarian cancer [43]. HOX genes are also involved in the activation of T lymphocytes and have been shown to be responsible for the immune response to ovarian carcinoma [44,45].

During development, HOX gene clusters are expressed in sequence along the chromosome as the spatial arrangement of the developing tissue progresses from anterior to posterior. This segmental polarity is seen in the mammalian female paramesonephric duct as it develops into the fallopian tube, uterus, cervix and upper vagina [29]. These structures develop according to the Müllerian pathway of differentiation and are characterized by highly structured epithelial architecture. HOX gene expression remains active in these tissues throughout adulthood and is thought to function in the maintenance of cell identity and the highly differentiated phenotype [46]. Arising from embryologic mesoderm, the ovary is not a part of this Müllerian system of development, and the normal ovarian surface epithelium maintains a highly undifferentiated structure. This down-regulation of HOXC6 in ovarian carcinoma seems to be unique in the HOX family of genes as multiple HOX genes are known to be up-regulated including HOXA7, HOXB2, and HOXB7 [47,48]. Loss of gene function is a known finding in cancer and can be directly related to clinical outcome [49]. Further examination of direct downstream targets of HOXC6 within these cells may define critical pathways in the development and maintenance of normal ovarian epithelium and shed light on their dysregulation following HOXC6 loss and tumor development.

The role of individual HOX genes as either oncogene or tumor suppressors is well established [5]. Similarly, HOXC6 regulates genes with both oncogenic and tumor suppressor activities. It may thus act as either a tumor suppressor or oncogene dependent on tissue context or additional cooperating mutations. Overexpression of HOXC6 has been demonstrated in the human malignancies meduloblastomas, osteosarcomas, breast, lung, gastrointestinal, head and neck squamous, and prostate carcinomas, leukemia as well as normal trophoblast [4, 23-27]. HOXC6 function may be modulated by a variety of secreted factors such as growth factors, cytokines, and hormones. Evidence that HOXC6 is regulated by TGF-beta suggests HOX genes are targets for the TGF-beta superfamily of genes and serve as a mechanism for growth factors to exert their effect on development and carcinogenesis [18]. Estradiol regulation of HOXC6 has specific implications to HOXC6 function in ovarian and Müllerian tissues. Ansari et al. [12-19] reported a dose dependent response of HOXC6 gene expression to estradiol in vitro. Changes in the estrogen micro- or macro-environment could affect the expression and role of HOXC6 in ovarian cancer tumorigenesis [19-20]. Although HOXC6 is over-expressed in hormone responsive breast and prostate tumors [13,50], it is notable that the observed elevated expression of HOXC6 in mammary glands of ovariectomized female mice suggests negative regulation of HOXC6 by ovarian hormones [51,52]. This finding supports the idea that HOXC6 expression is potentially both positively and negatively regulated by steroid hormones in a tissue-dependent manner. Hypermethylation in a number of tumors leads to loss of tumor suppressor function in multiple tumor types. Transcription of HOXC6 is repressed by hypermethylation via Lsh protein mediated Polymerase II stalling [53] and in association with MLL1 and MLL4 histone methyltransferase binding to the HOXC6 promoter region [38]. Elevated methylation associated with decreased gene expression has been observed in cases of high-grade serous ovarian cancer [54] although the specific status of HOXC6 promoter region has not been examined in this study.

Our finding of constitutively elevated levels of HOXC6 in normal ovarian epithelial cells and down-regulation in serous ovarian carcinoma, the most common ovarian malignancy, suggests a potential role as one biomarker for ovarian cancer. In a clinical oncology setting, biomarkers can have various functions including detecting tumors, monitoring response to treatment, or serving as therapeutic target. The decreased levels of HOXC6 in the serum of patients with ovarian cancer, shown in this study, is in stark contrast to the elevated levels of well-described biomarkers CA125 and HE4 [55,56]. The finding of HOXC6 down-regulation offers a change in the paradigm in that down-regulation of a gene specific to a malignancy may be important clinically in the context of detection as well as biologically in the process of oncogenesis. In our previous ovarian cancer microarray study, strengths of correlation and significance in gene expression between pairs of HOX family genes were highly variable, yet several patterns within subgroups are clear. HOXC6 demonstrated correlation with three IGFBP genes [28]. Additional studies of HOXC6 in combination with known serum biomarkers will better define the significance of our findings related to biomarker function. Further, regulation of the multiple drug resistance (MDR) genes by HOXC6 has been reported in vitro, and thus development of HOXC6 as a therapeutic target may have implications in chemotherapy-resistant ovarian cancer [21].

Our findings are the first characterization of HOXC6 gene and protein expression patterns in serous ovarian malignancy. Given that a growing body of evidence suggests an increasing number of ovarian cancers may in fact arise from the fallopian tube [57,58], understanding of the role of HOXC6 in ovarian carcinoma may be further enhanced by studies of HOXC6 expression patterns in fallopian tube and non-serous ovarian carcinomas. This is supported by our ELISA data containing a small cohort of samples from patients with fallopian tube carcinoma. Our finding of detectable levels of HOXC6 protein that are reduced in patients with high-grade serous ovarian carcinoma is significant.

TABLE 2
Characteristics of malignant samples used; All
samples are high-grade serous carcinoma.

| Sample | | | | | |
|---|---|---|---|---|---|
| # | Stage | Age | Collected Sample | Tumor site | Assay |
| M1 | III-B | 75 | Tissue & Serum | Ovary | Microarray (MA), |
| | | | | | ELISA |
| M2 | III-C | 54 | Tissue & Serum | Ovary | MA, IHC, ELISA |
| M3 | III-C | 74 | Tissue & Serum | Ovary | MA, qPCR, IHC, |
| | | | | | ELISA |
| M4 | III-C | 64 | Tissue & Serum | Ovary | MA, qPCR, IHC, |
| | | | | | ELISA |
| M5 | III-C | 54 | Tissue & Serum | Ovary | MA, qPCR, IHC, |
| | | | | | ELISA |
| M6 | III-C | 57 | Tissue & Serum | Ovary | MA, IHC, ELISA |
| M7 | III-C | 71 | Tissue & Serum | Ovary | MA, IHC, ELISA |
| M8 | III-C | 43 | Serum | Ovary | ELISA |
| M9 | III-C | 64 | Serum | Ovary | ELISA |
| M10 | III-C | 63 | Serum | Ovary | ELISA |
| M11 | III-C | 81 | Serum | Fallopian | ELISA |
| | | | | Tube | |
| M12 | IV | 75 | Serum | Ovary | ELISA |
| M13 | III-C | 69 | Serum | Fallopian | ELISA |
| | | | | Tube | |
| M14 | III-C | 62 | Serum | Fallopian | ELISA |
| | | | | Tube | |
| M15 | III-C | 62 | Serum | Fallopian | ELISA |
| | | | | Tube | |
| M16 | III-C | 81 | Serum | Ovary | ELISA |
| M17 | IV | 68 | Serum | Ovary | ELISA |
| M18 | III-C | 80 | Serum | Ovary | ELISA |
| M19 | IV | 75 | Serum | Fallopian | ELISA |
| | | | | Tube | |
| M20 | III-B | 44 | Serum | Ovary | ELISA |
| M21 | III-C | 76 | Serum | Ovary | ELISA |

TABLE 3
Up- and down-regulated HOX genes dysregulated in serous
ovarian carcinoma generated from microarray analysis.

| Gene name | Gene ID | p-value | Fold change |
|---|---|---|---|
| Down-regulated: | | | |
| HOXA4 | NM_002141 | 0.03483 | −1.3075 |
| HOXC6 | NM_004503 | 0.00364 | −2.1850 |
| HOXC9 | NM_006897 | 0.03245 | −1.8186 |
| HOXD8 | NM_019558 | 0.03529 | −1.7686 |
| Up-regulated: | | | |
| HOXB2 | NM_002145 | 0.00240 | 1.7423 |
| HOXB3 | NM_002146 | 0.01187 | 2.4289 |
| HOXB5 | NM_002147 | 0.03546 | 1.9590 |
| HOXB7 | NM_004502 | 0.03999 | 1.3667 |
| HOXB8 | NM_024016 | 0.00917 | 2.7503 |
| Not significant: | | | |
| HOXA10 | NM_018951 | 0.60377 | −1.1157 |
| HOXA13 | NM_000522 | 0.80213 | −1.0429 |
| HOXA2 | NM_006735 | 0.37518 | −1.1932 |
| HOXA3 | NM_153631 | 0.41751 | −1.1235 |
| HOXA5 | NM_019102 | 0.84787 | 1.0441 |
| HOXA6 | NM_024014 | 0.89826 | −1.0210 |
| HOXA7 | NM_006896 | 0.09294 | −1.3266 |
| HOXA9 | NM_152739 | 0.65671 | −1.0564 |
| HOXB1 | NM_002144 | 0.69206 | −1.0793 |
| HOXB13 | NM_006361 | 0.22251 | −1.2476 |
| HOXB4 | NM_024015 | 0.13328 | 1.2174 |
| HOXB6 | NM_018952 | 0.23631 | 1.7481 |
| HOXB9 | NM_024017 | 0.25668 | 1.3327 |
| HOXC10 | NM_017409 | 0.33679 | −1.1718 |
| HOXC11 | NM_014212 | 0.60687 | −1.1533 |
| HOXC12 | NM_173860 | 0.72553 | −1.0746 |
| HOXC13 | NM_017410 | 0.40956 | −1.1538 |
| HOXC8 | NM_022658 | 0.05016 | −1.4639 |
| HOXD1 | NM_024501 | 0.48361 | 1.2490 |
| HOXD10 | NM_002148 | 0.93087 | 1.0181 |
| HOXD11 | NM_021192 | 0.27080 | −1.2161 |
| HOXD12 | NM_021193 | 0.15516 | −1.3303 |
| HOXD13 | NM_000523 | 0.84913 | −1.0334 |
| HOXD3 | NM_006898 | 0.70621 | −1.2071 |
| HOXD4 | NM_014621 | 0.26843 | −1.3414 |
| HOXD9 | NM_014213 | 0.07467 | −1.5340 |
| HOXA1 | NM_005522 | 0.09329 | −1.3183 |
| HOXA11 | NM_005523 | 0.07397 | −1.3466 |

TABLE 4
Quantitative RT-PCR analysis of HOXC6 expression
in human epithelial cell lines.

| | | | | | HOXC6 | |
|---|---|---|---|---|---|---|
| | | | | | Fold | |
| # | Cell-line | From* | Origin | Type | Change | p-Value |
| 1 | HOSE | S.W. | Ovarian | Normal | N/A | N/A |
| | 6-3 | Tsao | Surface | Immortalized | | |
| | | [32] | epithelial | | | |
| 2 | SKOV-3 | ATCC | Ovarian | Adenocarcinoma | −12.5 | 0.016939552 |
| | (HTB77) | | epithelial | | | |
| 3 | SW626 | ATCC | Colon-to- | Metastatic | −2.80 | 0.058767875 |
| | (HTB78) | | Ovarian | Adenocarcinoma | | |
| | | | epithelial | (Grade III) | | |
| 4 | CaOv-3 | ATCC | Ovarian | Adenocarcinoma | −1.37 | 0.283791563 |
| | (HTB75) | | epithelial | | | |
| 5 | MDA-MB- | ATCC | Breast | Adenocarcinoma | 530 | 0.00998836 |
| | 231 | | epithelial | | | |
| | (HTB26) | | cell | | | |
| 6 | HeLa | ATCC | Cervix | Adenocarcinoma | 3.21 | 0.01114571 |
| | (CCL2) | | | | | |

*ATCC: American Type Culture collection.

### REFERENCES

1. Siegel R, Naishadham D, Jemal A: Cancer statistics, 2013. C A: a cancer journal for clinicians 2013, 63(1):11-30.
2. Flemming G., Siedman, Ernst L., Epithelial ovarian cancer. In: Barakat R, Berchuck A, Markman M, Randall M, eds. Principles and Practice of Gynecologic Oncology, 6^{th} ed. Philadelphia, Pa.: Lippincott Williams & Wilkins; 2013; 757-847.
3. Fekete T, Raso E, Pete I, Tegze B, Liko I, Munkacsy G, Sipos N, Rigo J, Jr., Gyorffy
B: Meta-analysis of gene expression profiles associated with histological classification and survival in 829 ovarian cancer samples. Int J Cancer 2012, 131(1):95-105.
4. Alexander T, Nolte C, Krumlauf R: Hox genes and segmentation of the hindbrain and axial skeleton. Annu Rev Cell Dev Biol 2009, 25:431-456.
5. Colleen D. McCabe, Demetri D. Spyropoulos, David Martin, and Carlos S. Moreno. Genome-Wide Analysis of the Homeobox C6 Transcriptional Network in Prostate Cancer. Cancer research. 2008. The AJCR: 1931-1940
6. Calvo R, West J, Franklin W, Erickson P, Bemis L, Li E, Helfrich B, Bunn P, Roche J, Brambilla E et al: Altered HOX and WNT7A expression in human lung cancer. Proceedings of the National Academy of Sciences of the United States of America 2000, 97(23):12776-12781.
7. Flagiello D, Poupon M F, Cillo C, Dutrillaux B, Malfoy B: Relationship between
DNA methylation and gene expression of the HOXB gene cluster in small cell lung cancers. FEBS Lett 1996, 380(1-2):103-107.
8. Hung Y C, Ueda M, Terai Y, Kumagai K, Ueki K, Kanda K, Yamaguchi H, Akise D, Ueki M: Homeobox gene expression and mutation in cervical carcinoma cells. Cancer Sci 2003, 94(5):437-441.
9. Lane D B, Rutherford T J, Taylor H S: HOXA10 expression in endometrial adenocarcinoma. Tumour Biol 2004, 25(5-6):264-269.
10. Segara D, Biankin A V, Kench J G, Langusch C C, Dawson A C, Skalicky D A, Gotley D C, Coleman M J, Sutherland R L, Henshall S M: Expression of HOXB2, a retinoic acid signaling target in pancreatic cancer and pancreatic intraepithelial neoplasia. Clin Cancer Res 2005, 11(9):3587-3596.
11. Takahashi Y, Hamada J, Murakawa K, Takada M, Tada M, Nogami I, Hayashi N, Nakamori S, Monden M, Miyamoto M et al: Expression profiles of 39 HOX genes in normal human adult organs and anaplastic thyroid cancer cell lines by quantitative real-time RT-PCR system. Exp Cell Res 2004, 293(1):144-153.
12. Chariot A, Castronovo V, Le P, Gillet C, Sobel M, Gielen J. Cloning and expression of a new HOXC6 transcript encoding a repressing protein. Biochem J, 1996; 319: 91-97.
13. Bodey B, Bodey B, Jr., Siegel S E, Kaiser H E: Immunocytochemical detection of the homeobox B3, B4, and C6 gene products in childhood medulloblastomas/primitive neuroectodermal tumors. Anticancer Res 2000, 20(3A):1769-1780.
14. Villaescusa J C, Verrotti A C, Ferretti E, Farookhi R, Blasi F: Expression of Hox cofactor genes during mouse ovarian follicular development and oocyte maturation. Gene 2004, 330:1-7.
15. Bodey B, Bodey B, Jr., Siegel S E, Kaiser H E: Immunocytochemical detection of the homeobox B3, B4, and C6 gene products within the human thymic cellular microenvironment. In Vivo 2000, 14(3):419-424.
16. Bill J J, van Oostveen J W, Walboomers J M, Brink A T, Vos W, Ossenkoppele G J, Meijer C J. Differentiation and cell-type-restricted expression of HOXC4, HOXC5 and HOXC6 in myeloid leukemias and normal myeloid cells. Leukemia 1998; 12: 1724-1732.
17. Garcia-Gasca A, Spyropoulos D D. Differential mammary morphogenesis along the anteroposterior axis in Hoxc6 gene targeted mice. Dev Dynam 2000; 219: 261-276.
18. Kloen P, Visker M H, Olijve W, van Zoelen E J, Boersma C J: Cell-type-specific modulation of Hox gene expression by members of the TGF-beta superfamily: a comparison between human osteosarcoma and neuroblastoma cell lines. Biochem Biophys Res Commun 1997, 233(2):365-369.
19. Ansari K I, Hussain I, Shrestha B, Kasiri S, Mandal S S: HOXC6 Is transcriptionally regulated via coordination of MLL histone methylase and estrogen receptor in an estrogen environment. J Mol Biol 2011, 411(2):334-349.
20. Singleton D W, Feng Y, Yang J, Puga A, Lee A V, Khan S A: Gene expression profiling reveals novel regulation by bisphenol-A in estrogen receptor-alpha-positive human cells. Environ Res 2006, 100(1):86-92.
21. Kim K J, Moon S M, Kim S A, et al. Transcriptional Regulation of MDR-1 by HOXC6 in Multidrug-resistant Cells. Oncogene, 2013; 32:3339-3349.
22. Anna Vinarskaja, Masanori Yamanaka, Marc Ingenwerth, and Wolfgang A. Schulz. DNA Methylation and the HOXC6 Paradox in Prostate Cancer Cancers (Basel). December 2011; 3(4): 3714-3725. Published online Sep. 27, 2011.
23. Bodey B, Bodey B, Jr., Siegel S E, Luck J V, Kaiser H E: Homeobox B3, B4, and C6 gene product expression in osteosarcomas as detected by immunocytochemistry. Anticancer Res 2000, 20(4):2717-2721. 23
24. Castronovo V, Kusaka M, Chariot A, Gielen J, Sobel M: Homeobox genes: potential candidates for the transcriptional control of the transformed and invasive phenotype. Biochem Pharmacol 1994, 47(1):137-143.
25. Fujiki K, Duerr E M, Kikuchi H, Ng A, Xavier R J, Mizukami Y, Imamura T, Kulke M H, Chung D C: Hoxc6 is overexpressed in gastrointestinal carcinoids and interacts with JunD to regulate tumor growth. Gastroenterology 2008, 135(3):907-916, 916 e901-902.
26. Miller G J, Miller H L, van Bokhoven A, Lambert J R, Werahera P N, Schirripa O, Lucia M S, Nordeen S K: Aberrant HOXC expression accompanies the malignant phenotype in human prostate. Cancer Res 2003, 63(18):5879-5888.
27. Moon S M, Kim S A, Yoon J H, Ahn S G: HOXC6 is deregulated in human head and neck squamous cell carcinoma and modulates Bcl-2 expression. The Journal of biological chemistry 2012, 287(42):35678-35688.
28. Bahrani-Mostafavi Z, Tickle T L, Zhang J, Bennett K E, Vachris J C, Spencer M D, Mostafavi M T, Tait D L: Correlation analysis of HOX, ErbB and IGFBP family gene expression in ovarian cancer. Cancer Invest 2008, 26(10):990-998.
29. Naora H, Montz F J, Chai C Y, Roden R B: Aberrant expression of homeobox gene HOXA7 is associated with mullerian-like differentiation of epithelial ovarian tumors and the generation of a specific autologous antibody response. Proceedings of the National Academy of Sciences of the United States of America 2001, 98(26):15209-15214.
30. Naora H, Yang Y Q, Montz F J, Seidman J D, Kurman R J, Roden R B: A serologically identified tumor antigen encoded by a homeobox gene promotes growth of ovarian epithelial cells. Proceedings of the National Academy of Sciences of the United States of America 2001, 98(7):4060-4065.
31. Tickle T L: Data mining the serous ovarian tumor transcriptome. Charlotte: University of North Carolina; 2011.
32. Tsao S W, Mok S C, Fey E G, Fletcher J A, Wan T S, Chew E C, Muto M G, Knapp R C, Berkowitz R S: Characterization of human ovarian surface epithelial cells immortalized by human papilloma viral oncogenes (HPV-E6E7 ORFs). Exp Cell Res 1995, 218(2):499-507.
33. Schmittgen T D, Livak K J: Analyzing real-time PCR data by the comparative C(T) method. Nat Protoc 2008, 3(6):1101-1108.
34. Bahrani Z, Sherwood J, Sanborn M, Keyser G: The Use of Monoclonal Antibodies to detect wheat soil-borne mosaic virus. J Gen Virol 1988, 69:1317-1322.6d
35. Eisenhart C: The assumptions underlying the analysis of variance. Biometrics 1947, 3(1):1-22.
36. Tamhane A, Dunlop D: Statistics and data analysis: from elementary to intermediate: Prentice Hall; 2000.
37. Steller M D, Shaw T J, Vanderhyden B C, Ethier J F. Inhibin resistance is associated with aggressive tumorigenicity of ovarian cancer cells. Mol Cancer Res. 2005 January; 3(1):50-61.
38. Bai F, Feng J, Cheng Y, Shi J, Yang R, Cui H. Analysis of gene expression patterns of ovarian cancer cell lines with different metastatic potentials. Int J Gynecol Cancer. 2006 January-February; 16(1):202-9.
39. Furlong M T, Hough C, Sherman-Baust C A, Pizer E S, Morin P I, Evidence for the Colonic Origin of Ovarian Cancer Cell Line SW626 J Nat. Cancer Inst. 1999, 91(15) 1327-8.
40. Care A, Felicetti F, Meccia E, Bottero L, Parenza M, Stoppacciaro A, Peschle C, Colombo M P: HOXB7: a key factor for tumor-associated angiogenic switch. Cancer Res 2001, 61(17):6532-6539.
41. Care A, Silvani A, Meccia E, Mattia G, Stoppacciaro A, Parmiani G, Peschle C, Colombo M P: HOXB7 constitutively activates basic fibroblast growth factor in melanomas. Mol Cell Biol 1996, 16(9):4842-4851.
42. Ono R, Kumagai H, Nakajima H, Hishiya A, Taki T, Horikawa K, Takatsu K, Satoh T, Hayashi Y, Kitamura T et al: Mixed-lineage-leukemia (MLL) fusion protein collaborates with Ras to induce acute leukemia through aberrant Hox expression and Raf activation. Leukemia 2009, 23(12):2197-2209.
43. Burger R A, Brady M F, Bookman M A, Fleming G F, Monk B J, Huang, H., Mannel R S, Homesley H D, Flower J, Greer B E, Boente M, Birrer M J, and Liang S X., Incorporation of Bevacizumab in the Primary Treatment of Ovarian Cancer, NEJM 2011; 265:2473-83.
44. Care A, Testa U, Bassani A, Tritarelli E, Montesoro E, Samoggia P, Cianetti L, Peschle C: Coordinate expression and proliferative role of HOXB genes in activated adult T lymphocytes. Mol Cell Biol 1994, 14(7):4872-4877.
45. Conejo-Garcia J R, Benencia F, Courreges M C, Gimotty P A, Khang E, Buckanovich R J, Frauwirth K A, Zhang L, Katsaros D, Thompson C B et al: Ovarian carcinoma expresses the NKG2D ligand Letal and promotes the survival and expansion of CD28-antitumor T cells. Cancer Res 2004, 64(6):2175-2182.
46. Cheng W, Liu J, Yoshida H, Rosen D, Naora H: Lineage infidelity of epithelial ovarian cancers is controlled by HOX genes that specify regional identity in the reproductive tract. Nat Med 2005, 11(5):531-537.
47. Tait D, McKinney K, Zhang J, Vachris J C, Gurlov S, Hall J, Higgins R, MNaumann R, Bahra-Mostafavi Z: Up-regulation of HOXB7 gene in epithelial ovarian cancer. Proceedings of the Thirty-seventh annual meeting of the Society of Gynecologic Oncologists 2006.
48. Tait D, Zhang J, Gurlov S, McKinney K, Vachris J C, Mongeot J, Wagstaff J, Bahra-Mostafavi Z: Differential expression of HOX genes in ovarian cancer. Proceedings of teh Ninety-sixth annual meeting of the American Association for Cancer Research 2005.
49. Konstantinopoulos, Panagiotis A; Spentzos, Dimitrios; Karlan, Beth Y; Taniguchi, Toshiyasu, Fountzilas, Elena; Francoeur, Nancy; Levine, Douglas A; Cannistra, Stephen A. Gene expression profile of BRCAness that correlates with responsiveness to chemotherapy and with outcome in patients with epithelial ovarian cancer, Journal of Clinical Oncology, 2010; 28: 3555-61.
50. Ramachandran S, Liu P, Young A N, Yin-Goen Q, Lim S D, Laycock N, Amin M B, Carney J K, Marshall F F, Petros J A et al: Loss of HOXC6 expression induces apoptosis in prostate cancer cells. Oncogene 2005, 24(1):188-198.
51. Duverger 0, Morasso M I: Role of homeobox genes in the patterning, specification, and differentiation of ectodermal appendages in mammals. J Cell Physiol 2008, 216(2):337-346.
52. Friedmann Y, Daniel C A, Strickland P, Daniel C W: Hox genes in normal and neoplastic mouse mammary gland. Cancer Res 1994, 54(22):5981-5985.
53. Tao Y, Xi S, Briones V, Muegge K: Lsh mediated RNA polymerase II stalling at HoxC6 and HoxC8 involves DNA methylation. PLoS One 2010, 5(2):e9163.
54. Integrated genomic analyses of ovarian carcinoma. Nature 2011, 474(7353):609-615.
55. Markman M, Liu P Y, Rothenberg M I, et al. Pre-treatment C A-125 and risk of relapse in advanced ovarian cancer, 2006, J Clin Oncol; 24:1454-1458.
56. Cheng X, Ye X, Dong L, et al. Human Epididymis Protein 4 (HE$) as a Serum Tumor Biomarker in Patients with Ovarian Cancer. Int J. Gyncol Cancer 2011; 21:852-858.
57. Przybycin C, Kurman R J, Ronnett B M, Shih I, Vang R. Are all pelvic (nonuterine) serous carcinomas of tubal origin?, Am J Surg Path 2010; 34:1407-1416.
58. Auersperg N. The origin of ovarian carcinomas: a unifying hypothesis. Int J Gynecol Path 2010; 30:12-21.

It is to be understood that, while the invention has been described in conjunction with the detailed description, thereof, the foregoing description is intended to illustrate and not limit the scope of the invention. Other aspects, advantages, and modifications of the invention are within the scope of the claims set forth below. All publications, patents, and patent applications cited in this specification are herein incorporated by reference as if each individual publication or patent application were specifically and individually indicated to be incorporated by reference.

What is claimed is:

1. A method for detecting the likelihood of ovarian cancer which comprises:
measuring a level of HOXC6 in a blood or tissue sample; and
if the level of HOXC6 is 50% or less than a normal level of HOXC6, determining that the blood or tissue sample indicates an increased likelihood of having ovarian cancer.

2. The method of claim 1, wherein the level of HOXC6 is measured using an antibody-based assay.

3. The method of claim 2, wherein the antibody based assay is an indirect enzyme-linked immunosorbent assay (ELISA).

4. The method of claim 1, wherein the blood sample is a blood serum sample.

5. A kit comprising: at least one reagent selected from the group consisting of:
a primary antibody capable of specifically binding a HOXC6 protein;
a secondary antibody capable of detecting the primary antibody bound to the HOXC6 protein; and
instructions for use in measuring a level of HOXC6 from a subject suspected of having ovarian cancer wherein levels of 50% or less than a normal level, determining that the subject has increased likelihood of having ovarian cancer.

6. A method of identifying a compound that may become a therapeutic target for the treatment of ovarian cancer, the method comprising the steps of:
contacting a compound with a sample comprising a cell or a tissue;
measuring a level of HOXC6 in the cell or tissue; and
determining a functional effect of the compound on the level of HOXC6; thereby
identifying a compound that prevents or treats ovarian cancer.
